(19) United States
(12) Patent Application Publication
(10) Pub. No.: US 2008/0051400 A1
(43) Pub. Date: Feb. 28, 2008

(54) Methods for treating or preventing anemia or thrombocytopenia using a triheterocyclic compound

(75) Inventors: Jean Viallet, Malvern, PA (US); Susan Mary O'Brien, Houston, TX (US)

Correspondence Address: WILSON SONSINI GOODRICH & ROSATI, 650 PAGE MILL ROAD, PALO ALTO, CA, 94304-1050, US

(73) Assignee: Gemin X Biotechnologies Inc., Montreal (CA)

(21) Appl. No.: 11/825,194

(22) Filed: Jul. 5, 2007

Related U.S. Application Data
(60) Provisional application No. 60/818,781, filed on Jul. 6, 2006.

(30) Foreign Application Priority Data
Oct. 10, 2006 (CA) 2,563,116

(51) Int. Cl. A61K 31/4025; A61K 31/497; A61K 31/5377; A61P 7/02; A61P 7/06

(52) U.S. Cl. 514/235.2; 514/254.09; 514/414

(57) ABSTRACT

The present invention relates to methods useful for treating or preventing anemia or thrombocytopenia, comprising administrating an effective amount of a Triheterocyclic Compound to a subject in need thereof. The present invention also relates to methods useful for preventing anemia or thrombocytopenia in a subject, comprising administrating an effective amount of a Triheterocyclic Compound to the subject, wherein the subject is at heightened risk of developing anemia or thrombocytopenia.

This application claims the benefit of U.S. provisional application No. 60/818,781, filed Jul. 6, 2006, which is incorporated by reference herein in its entirety.

### 1. FIELD OF THE INVENTION

The present invention relates to methods useful for treating or preventing anemia or thrombocytopenia comprising administrating an effective amount of a Triheterocyclic Compound to a subject in need thereof. The present invention also relates to methods useful for preventing anemia or thrombocytopenia in a subject, comprising administrating an effective amount of a Triheterocyclic Compound to the subject, wherein the subject is at heightened risk of developing anemia or thrombocytopenia.

### 2. BACKGROUND OF THE INVENTION

Disorders of the blood present many important health concerns. For example low number of red blood cells, decreased volume of red blood cells, or reduced hemoglobin concentration can be indicative of pathological conditions such as, but not limited to, anemia. The symptoms of anemia may include fatigue, dizziness, headache, chest pain, shortness of breath, and depression. For certain forms of anemia, medications and even chemotherapy are used in the treatment or management of the disorder. Various examples of medications include antibiotics, immune-suppressing medication, pain-relieving medication and antimetabolites such as hydroxyurea (Droxia®and Hydrea®).

Another example of a blood disorder is thrombocytopenia. Platelets or thrombocytes are also found in blood cells and are involved in the cellular mechanisms that lead to the formation of blood clots. Low platelet counts increase bleeding risks and can occasionally be treated by intranasal desmopressin (ddAVP).

In both cases, abnormally low hemoglobin or platelet counts can be treated through blood transfusions or in very severe cases, through bone marrow transfers. These procedures, however, can have associated risks. For example, there may be difficulties in finding a matching bone marrow donor. Furthermore, transfusion reactions may occur if the blood types are not properly matched between donor and patient. There is also a risk of infection from viruses and other pathogens from the donor blood. There is also a concern that frequent blood transfusions may damage the immune system, which protects the body from infections.

With respect to anemia, some regulatory authorities have approved recombinant human erythropoietin for treatment of anemia associated with chronic renal failure (CRF), anemia related to therapy with AZT (zidovudine) in HIV-infected patients, anemia in patients having non-myeloid malignancies receiving chemotherapy, and anemia in patients undergoing surgery to reduce the need of allogenic blood transfusions.

Examples of various treatments to increase or maintain hematocrit or platelet count in a mammal are disclosed in International Publication No. WO 00/24893; U.S. Pat. No. 6,956,022; and U.S. Patent Application Publication Nos. 2005-0106148 and 2003-0215444.

Due to the serious health problems arising from blood disorders, such as those associated with low hemoglobin or platelet counts, it is desirable of developing other agents capable of treating these disorders.

All documents cited in this Application are incorporated by reference herein for all purposes.

### 3. SUMMARY OF THE INVENTION

In one embodiment, the present invention provides methods for treating or preventing anemia or thrombocytopenia, comprising administering to a subject in need thereof an effective amount of a compound having the Formula (Ia): 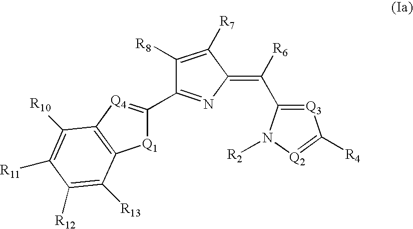
or a pharmaceutically acceptable salt thereof, wherein:

Q₁ is —O—, —S— or —N(R₁)—;

Q₂ is —C(R₃)— or —N—;

Q₃ is —C(R₅)— or —N—;

Q₄ is —C(R₉)— or —N—;

R₁ is —Yₘ(Rₐ), wherein —Rₐ is —H, —OH, —C₁-C₈ alkyl, —C₂-C₈ alkenyl, —C₂-C₈ alkynyl, —C₃-C₁₂ cycloalkyl, -phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —OS(O)₂OH, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, or —NR₁₄C(S)N(R₁₄)₂;

R₂ is —H, —C₁-C₈ alkyl or —OH;

R₃, R₄, and R₅ are independently —Yₘ(R_{b}), wherein R_{b} is —H, halogen, —NH₂, —CN, —NO₂, —SH, —N₃, —C₁-C₈ alkyl, —O—(C₁-C₈ alkyl), —C₂-C₈ alkenyl, —C₂-C₈ alkynyl, —C₃-C₁₂ cycloalkyl, -phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O(CH₂)ₙ—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂ or R₃ and R₄, or R₄ and R₅, together with the carbon atom to which each is attached, join to form a 5- to 9-membered ring, with the proviso that if Q₃ is —C(R₅)— and m=0, then R₅ is not H;

R₆ is —H, halogen, —OH, —NH₂, —C₁-C₈ alkyl, or —O—(C₁-C₈ alkyl);

R₇ is —Yₘ—(R_{c}), wherein —R_{c} is —C₁-C₈ alkyl, —O—(C₁-C₈ alkyl), —O-benzyl, —OH, —NH₂, —NH(C₁-C₅ alkyl), —N(C₁-C₅ alkyl)₂, —NH(phenyl), —N(phenyl)₂, —NH(naphthyl), —N(naphthyl)₂, —CN, —NO₂, —N₃, —C₂-C₈ alkynyl, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, O(CH₂)ₙC(O)O(CH₂)ₙCH₃, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂;

R₈ is —Yₘ(R_{d}), wherein —R_{d} is —H, —OH, halogen, —NH₂, —NH(C₁-C₅ alkyl), —N(C₁-C₅ alkyl)₂, —NH(phenyl), —N(phenyl)₂, —NH(naphthyl), —N(naphthyl)₂, —CN, —NO₂, —N₃, —C₁-C₈ alkyl, —O—(C₁-C₈ alkyl), —(C₁-C₈ alkylene)-OH, —C₂-C₈ alkenyl, —C₂-C₈ alkynyl, —C₃-C₁₂ cycloalkyl, —phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂;

R₉, R₁₀, R₁₁, R₁₂, and R₁₃ are independently —Yₘ(Rₑ), wherein —Rₑ is —H, halogen, —NH₂, C₁-C₈ alkyl, —NH(C₁-C₅ alkyl), —N(C₁-C₅ alkyl)₂, —NH(C₂-C₅ alkenyl), —N(C₂-C₅ alkenyl)₂, —NH(phenyl), —N(phenyl)₂, —NH(naphthyl), —N(naphthyl)₂, —C(O)NH(C₁-C₅ alkyl), —C(O)N(C₁-C₅ alkyl)₂, —NHC(O)(C₁-C₅ alkyl), —NHC(═NH₂⁺)NH₂, —CN, —NO₂, N₃, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —C(O)C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂ or R₁₁ and R₁₂, together with the carbon atom to which each is attached, join to form a 5- to 9-membered ring;

each R₁₄ is independently —H, —C₁-C₈ alkyl, —C₃-C₁₂ cycloalkyl, -phenyl, -naphthyl, -3- to 9-membered heterocycle, —C₂-C₈ alkenyl, or —C₂-C₈ alkynyl;

each Y is independently —C₁-C₈ alkylene-, —C₂-C8 alkenylene- or —C₂-C₈ alkynylene-;

each m is independently 0 or 1;

each n is independently an integer ranging from 0 to 6; and

each p is independently an integer ranging from 1 to 6.

The invention further provides methods for treating or preventing anemia or thrombocytopenia, comprising administering to a subject in need thereof an effective amount of a compound having the Formula (Ib):

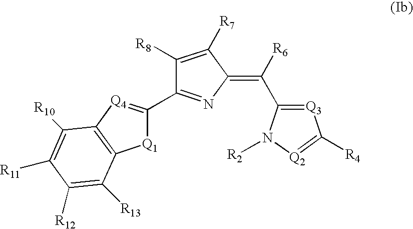

or a pharmaceutically acceptable salt thereof, wherein:

Q₁ is —O—, —S— or —N(R₁)—;

Q₂ is —C(R₃)— or —N—;

Q₃ is —C(R₅)— or —N—;

Q₄ is —C(R₉)— or —N—;

R₁ is —Yₘ(Rₐ), wherein —Rₐ is —H, —OH, —C₁-C₈ alkyl, —C₂-C₈ alkenyl, —C₂-C₈ alkynyl, —C₃-C₁₂ cycloalkyl, -phenyl, -naphthyl, - 3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —OS(O)₂OH, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, or —NR₁₄C(S)N(R₁₄)₂;

R₂ is —H, —C₁-C₈ alkyl or —OH;

R₃, R₄, and R₅ are independently —Yₘ(R_{b}), wherein R_{b} is —H, halogen, —NH₂, —CN, —NO₂, —SH, —N₃, C₁-C₈ alkyl, —O—(C₁-C₈ alkyl), —C₂-C₈ alkenyl, —C₂-C₈ alkynyl, —C₃-C₁₂ cycloalkyl, —phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O(CH₂)ₙ—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂ or R₃ and R₄, or R₄ and R₅ together with the carbon atom to which each is attached, join to form a 5- to 9-membered ring, with the proviso that if Q₃ is —C(R₅)— and m=0, then R₅ is not H;

R₆ is —H, halogen, —OH, —NH₂, —C₁-C₈ alkyl, or —O—(C₁-C₈ alkyl);

R₇ and R₈ are independently —Yₘ(R_{d}) wherein R_{d} is —H, —OH, halogen, —NH₂, —NH(C₁-C₅ alkyl), —N(C₁-C₅ alkyl)₂, —NH(phenyl), —N(phenyl)₂, —NH(naphthyl), —N(naphthyl)₂, —CN, —NO₂, —N₃, —C₁-C₈ alkyl, —O—(C₁-C₈ alkyl), —(C₁-C₈ alkylene)-OH, —O-benzyl, —C₂-C₈ alkenyl, —C₂-C₈ alkynyl, —C₃-C₁₂ cycloalkyl, -phenyl, -naphthyl, -3- to 9-membered heterocycle, —OR₁₄, —CH₂O(CH₂)ₚOR₁₄, —O—C(O)R₁₄, —C(O)(CH₂)ₙ—R₁₄, —C(O)R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂;

R₉, R₁₀, R₁₁, R₁₂, and R₁₃ are independently —Yₘ(Rₑ) wherein Rₑ is —H, halogen, —NH₂, C₁-C₈ alkyl, —NH(C₁-C₅ alkyl), —N(C₁-C₅ alkyl)₂, —NH(C₂-C₅ alkenyl), —N(C₂-C₅ alkenyl)₂, —NH(phenyl), —N(phenyl)₂, —NH(naphthyl), —N(naphthyl)₂, —C(O)NH(C₁-C₅ alkyl), —C(O)N(C₁-C₅ alkyl)₂, —NHC(O)(C₁-C₅ alkyl), —NHC(═NH₂⁺)NH₂, —CN, —NO₂, N₃, -3- to 9-membered heterocycle, —OR₁₄, —CH₂O(CH₂)ₚOR₁₄, —O—C(O)R₁₄, —C(O)(CH₂)ₙ—R₁₄, —C(O)R₁₄, —O—C(O)OR₁₄, —C(O)C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, -NR₁₄C(S)N(R₁₄)₂ or R₁₁ and R₁₂, together with the carbon atom to which each is attached, join to form a 5- to 9-membered ring;

each R₁₄ is independently —H, —C₁-C₈ alkyl, —C₃-C₁₂ cycloalkyl, -phenyl, -naphthyl, -3- to 9-membered heterocycle, —C₂-C₈ alkenyl, or —C₂-C₈ alkynyl;

each Y is independently —C₁-C₈ alkylene-, —C₂-C₈ alkenylene- or —C₂-C₈ alkynylene-;

each m is independently 0 or 1;

each n is independently an integer ranging from 0 to 6; and

each p is independently an integer ranging from 1 to 6.

The invention further provides methods for treating or preventing anemia or thrombocytopenia, comprising administering to a subject in need thereof an effective amount of a compound having the Formula (II):

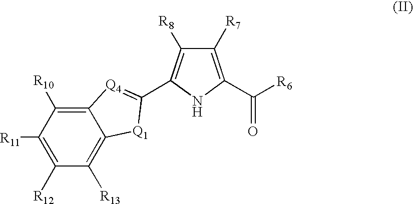

or a pharmaceutically acceptable salt thereof, wherein:

Q₁ is —O—, —S— or —N(R₁)—;

Q₄ is —C(R₉)— or —N—;

R₁ is —Yₘ(Rₐ), wherein —Rₐ is —H, —OH, —C₁-C₈ alkyl, —C₂-C₈ alkenyl, —C₂-C₈ alkynyl, —C₃-C₁₂ cycloalkyl, -phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —OS(O)₂OH, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, or —NR₁₄C(S)N(R₁₄)₂;

R₆ is —H, —OH, —NH₂, —C₁-C₈ alkyl, or —O—(C₁-C₈ alkyl);

R₇ and R₈ are independently —Yₘ(R_{d}) wherein R_{d} is —H, —OH, halogen, —NH₂, —NH(C₁-C₅ alkyl), —N(C₁-C₅ alkyl)₂, —NH(phenyl), —N(phenyl)₂, —NH(naphthyl), —N(naphthyl)₂, —CN, —NO₂, —N₃, —C₁-C₈ alkyl, —O—(C₁-C₈ alkyl), —(C₁-C₈ alkylene)-OH, —O-benzyl, —C₂-C₈ alkenyl, —C₂-C₈ alkynyl, —C₃-C₁₂ cycloalkyl, -phenyl, -naphthyl, —C₇-C₁₂ (phenyl)alkyl, —C₁₁-C₁₆ (naphthyl)alkyl, —C₈-C₁₂ (phenyl)alkenyl, —C₁₂-C₁₆ (naphthyl)alkenyl, —C₈-C₁₂ (phenyl)alkynyl, —C₁₂-C₁₆ (naphthyl)alkynyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂;

R₉, R₁₀, R₁₁, R₁₂, and R₁₃ are independently —Yₘ(Rₑ) wherein Rₑ is —H, halogen, —NH₂, C₁-C₈ alkyl, —NH(C₁-C₅ alkyl), —N(C₁-C₅ alkyl)₂, —NH(C₂-C₅ alkenyl), —N(C₂-C₅ alkenyl)₂, —NH(phenyl), —N(phenyl)₂, —NH(naphthyl), —N(naphthyl)₂, —C(O)NH(C₁-C₅ alkyl), —C(O)N(C₁-C₅ alkyl)₂, —NHC(O)(C₁-C₅ alkyl), —NHC(═NH₂⁺)NH₂, —CN, —NO₂, N₃, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —C(O)C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂ or R₁₁ and R₁₂, together with the carbon atom to which each is attached, join to form a 5- to 9-membered ring;

each R₁₄ is independently —H, —C₁-C₈ alkyl, —C₃-CI₂ cycloalkyl, -phenyl, -naphthyl, -3- to 9-membered heterocycle, —C₂-C₈ alkenyl, or —C₂-C₈ alkynyl;

each Y is independently —C₁-C₈ alkylene-, —C₂-C₈ alkenylene- or —C₂-C₈ alkynylene-;

each m is independently 0 or 1;

each n is independently an integer ranging from 0 to 6; and

each p is independently an integer ranging from 1 to 6.

The invention further provides methods for treating or preventing anemia or thrombocytopenia, comprising administering to a subject in need thereof an effective amount of a compound having the Formula (Ic):

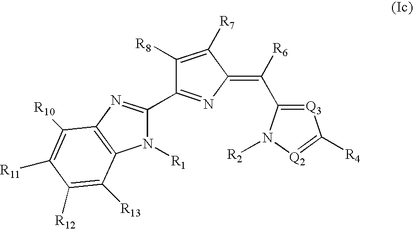

or a pharmaceutically acceptable salt thereof, wherein:

Q₂ is —C(R₃)— or —N—;

Q₃ is —C(R₅)— or —N—;

R₁ is —Yₘ(Rₐ), wherein —Rₐ is —H, —OH, —C₁-C₈ alkyl, —C₂-C₈ alkenyl, —C₂-C₈ alkynyl, —C₃-C₁₂ cycloalkyl, -phenyl, -naphthyl, - 3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —OS(O)₂OH, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, or —NR₁₄C(S)N(R₁₄)₂;

R₂ is —H, —C₁-C₈ alkyl or —OH;

R₃, R₄, and R₅ are independently —Yₘ(R_{b}), wherein R_{b} is —H, halogen, —NH₂, —CN, —NO₂, —SH, —N₃, —C₁-C₈ alkyl, —O—(C₁-C₈ alkyl), —C₂-C₈ alkenyl, —C₂-C₈ alkynyl, —C₃-C₁₂ cycloalkyl, —phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O(CH₂)ₙ—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂ or R₃ and R₄, or R₄ and R₅ together with the carbon atom to which each is attached, join to form a 5- to 9-membered ring;

R₆ is —H, halogen, —OH, —NH₂, —C₁-C₈ alkyl, or —O—(C₁-C₈ alkyl);

R₇ is —Yₘ—(R_{c}), wherein —R_{c} is —C₁-C₈ alkyl, —O—(C₁-C₈ alkyl), —O-benzyl, —OH, —NH₂, —NH(C₁-C₅ alkyl), —N(C₁-C₅ alkyl)₂, —NH(phenyl), —N(phenyl)₂, —NH(naphthyl), —N(naphthyl)₂, —CN, —NO₂, —N₃, —C₂-C₈ alkynyl, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O(CH₂)ₙC(O)O(CH₂)ₙCH₃, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂;

R₈ is —Yₘ(R_{d}), wherein —R_{d} is —H, —OH, halogen, —NH₂, —NH(C₁-C₅ alkyl), —N(C₁-C₅ alkyl)₂, —NH(phenyl), —N(phenyl)₂, —NH(naphthyl), —N(naphthyl)₂, —CN, —NO₂, —N₃, —C₁-C₈ alkyl, —O—(C₁-C₈ alkyl), —(C₁-C₈ alkylene)-OH, —C₂-C₈ alkenyl, —C₂-C₈ alkynyl, —C₃-C₁₂ cycloalkyl, —phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂;

R₁₀, R₁₁, R₁₂, and R₁₃ are independently —Yₘ(Rₑ), wherein —Rₑ is —H, halogen, —NH₂, C₁-C₈ alkyl, —NH(C₁-C₅ alkyl), —N(C₁-C₅ alkyl)₂, —NH(phenyl), —N(phenyl)₂, —NH(naphthyl), —N(naphthyl)₂, —C(O)NH(C₁-C₅ alkyl), —C(O)N(C₁-C₅ alkyl)₂, —NHC(O)(C₁-C₅ alkyl), —NHC(═NH₂⁺)NH₂, —CN, —NO₂, N₃, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂; or R₁₁ and R₁₂, together with the carbon atom to which each is attached, join to form a 5- to 9-membered ring;

each R₁₄ is independently —H, —C₁-C₈ alkyl, —C₃-C₁₂ cycloalkyl, -phenyl, -naphthyl, -3- to 9-membered heterocycle, —C₂-C₈ alkenyl, or —C₂-C₈ alkynyl;

each Y is independently —C₁-C₈ alkylene-, —C₂-C₈ alkenylene- or —C₂-C₈ alkynylene-;

each m is independently 0 or 1;

each n is independently an integer ranging from 0 to 6; and

each p is independently an integer ranging from 1 to 6.

The invention further provides methods for treating or preventing anemia or thrombocytopenia, comprising administering to a subject in need thereof an effective amount of a Prodigiosin Compound.

As used herein, a compound of Formula (Ia), (Ib), (II), (Ic), a Prodigiosin Compound, or a pharmaceutically acceptable salt thereof is a “Triheterocyclic Compound.”

A Triheterocyclic Compound is useful for treating or preventing anemia or thrombocytopenia in a subject. A Triheterocyclic Compound is useful for preventing anemia or thrombocytopenia in a subject, wherein the subject is at heightened risk of developing anemia or thrombocytopenia.

The details of the invention are set forth in the accompanying description below.

### 4. BRIEF DESCRIPTION OF THE DRAWINGS

FIG. 1 shows the evolution of peripheral blood parameters in patients having anemia or thrombocytopenia at baseline, before and after administration of the mesylate salt of Compound 1.

FIG. 2A shows hemoglobin counts in three patients having myelodysplastic syndromes (MDS) after administration of the mesylate salt of Compound 1 at 7, 14, and 40 mg/m² (respectively).

FIG. 2B shows platelets counts in three patients having myelodysplastic syndromes (MDS) after administration of the mesylate salt of Compound 1 at 7, 14, and 40 mg/m² (respectively). *Achievement of transfusion independence.

### 5. DETAILED DESCRIPTION OF THE INVENTION

### 5.1. Definitions and Abbreviations

As used herein and unless otherwise indicated, the term “anemia of chronic diseases” (ACD) refers to any anemia that is associated with a chronic disease persisting for more than two months, such as, but not limited to, trauma, infectious inflammation, non-infectious inflammation (e.g. rheumatoid arthritis (RA) or inflammatory bowel disease (IBD), lupus (including systemic lupus erythematosus or SLE), multiple sclerosis (MS), congestive heart failure (CHF), cardiovascular inflammation, and a neoplastic disease.

As used herein, the term “effective amount” is an amount that is effective to treat or prevent anemia or thrombocytopenia.

The phrase “pharmaceutically acceptable salt(s),” as used herein includes, but is not limited to, salts of acidic or basic groups that may be present in the Triheterocyclic Compound. Triheterocyclic Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, ethanesulfonate, benzenesulfonate, p-toluenesulfonate, mesylate, hydroxyethyl sulfonate, and pamoate (i.e., 1,1′-methylene-bis-(2-hydroxy-3-naphthoate)) salts. Triheterocyclic Compounds that include an amino moiety may form pharmaceutically acceptable salts with various amino acids, in addition to the acids mentioned above. Compounds that are acidic in nature are capable of forming base salts with various pharmacologically or cosmetically acceptable cations. Examples of such salts include alkali metal or alkaline earth metal salts and, particularly, calcium, magnesium, sodium, lithium, zinc, potassium, and iron salts.

In one embodiment in the methods for preventing anemia or thrombocytopenia, the subject has a heightened risk of developing anemia or thrombocytopenia. As used herein, the term “heightened risk” means that a subject is more likely to contract anemia or thrombocytopenia than a subject having an average risk. For example, the subject may have suffered from anemia or thrombocytopenia in the past, and be at risk of a relapse, or may exhibit symptoms which demonstrate to the practitioner that the subject is under an abnormal risk of developing anemia or thrombocytopenia.

As used herein, “halogen” refers to —F, —Cl, —Br or —I.

As used herein, “lower alkyl” or “C₁-C₆ alkyl” refers to a straight or branched chain saturated hydrocarbon group containing 1-6 carbon atoms which can be unsubstituted or optionally substituted with one or more -halogen, —NH₂, —OH, —O—(C₁-C₆ alkyl), phenyl or naphthyl groups. Examples of C₁-C₆ straight or branched chain alkyl groups include, but are not limited to, methyl, ethyl, 1-propyl, 2-propyl, 1-butyl, 2-butyl, 2-methyl-1-propyl, 2-methyl-2-propyl, 1-pentyl, 2-pentyl, 3-pentyl, 2-methyl-1-butyl, 3-methyl-1-butyl, 2-methyl-3-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl and 2-ethyl-1-butyl.

As used herein, “C₁-C₈ alkyl” refers to a straight or branched chain saturated hydrocarbon group containing 1-8 carbon atoms which can be unsubstituted or optionally substituted with one or more -halogen, —NH₂, —OH, —O—(C₁-C₈ alkyl), phenyl or naphthyl groups. Examples of C₁-C₈ straight or branched chain alkyl groups include, but are not limited to, methyl, ethyl, 1-propyl, 2-propyl, 1-butyl, 2-butyl, 2-methyl-1-propyl, 2-methyl-2-propyl, 1-pentyl, 2-pentyl, 3-pentyl, 2-methyl-1-butyl, 3-methyl-1-butyl, 2-methyl-3-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, 1-heptyl and 1-octyl.

As used herein, “C₁-C₅ alkyl” refers to a straight or branched chain saturated hydrocarbon group containing 1-5 carbon atoms. Examples of C₁-C₅ straight or branched chain alkyl groups include, but are not limited to, methyl, ethyl, 1-propyl, 2-propyl, 1-butyl, 2-butyl, 2-methyl-1-propyl, 2-methyl-2-propyl, 1-pentyl, 2-pentyl, 3-pentyl, 2-methyl-1-butyl, 3-methyl-1-butyl, 2-methyl-3-butyl, 2,2-dimethyl-1-propyl and 1-pentyl.

As used herein, “C₂-C₅ alkenyl” refers to an unsaturated, straight or branched chain hydrocarbon group containing 2-5 carbon atoms and at least one double bond.

As used herein, “C₂-C₈ alkenyl” refers to an unsaturated, straight or branched chain hydrocarbon group containing 2-8 carbon atoms and at least one double bond which can be unsubstituted or optionally substituted with a phenyl or naphthyl group.

As used herein, “C₂-C₈ alkynyl” refers to an unsaturated, straight or branched chain hydrocarbon group containing 2-8 carbon atoms and at least one triple bond which can be unsubstituted or optionally substituted with a phenyl or naphthyl group.

As used herein, “C₁-C₈ alkylene” refers to a C₁-C₈ alkyl group in which one of the C₁-C₈ alkyl group's hydrogen atoms has been replaced with a bond.

As used herein, “C₂-C₈ alkenylene” refers to a C₂-C₈ alkenyl group in which one of the C₂-C₈ alkenyl group's hydrogen atoms has been replaced with a bond.

As used herein, “C₂-C₈ alkynylene” refers to a C₂-C₈ alkynyl group in which one of the C₂-C₈ alkynyl group's hydrogen atoms has been replaced with a bond.

As used herein, “C₃-C₁₂ cycloalkyl” refers to a non-aromatic, saturated monocyclic, bicyclic or tricyclic hydrocarbon ring system containing 3-12 carbon atoms. Examples of C₃-C₁₂ cycloalkyl groups include, but are not limited to, cyclopropyl, cyclobutyl, cyclopentyl, cyclohexyl, cycloheptyl, cyclooctyl, norbornyl, adamantyl, bicyclo[2.2.2]oct-2-enyl, and bicyclo[2.2.2]octyl.

As used herein, a “C₇-C₁₂ (phenyl)alkyl” is a C₁-C₆ alkyl group substituted with a phenyl group.

As used herein, a “C₈-C₁₂ (phenyl)alkenyl” is a C₂-C₆ alkenyl group substituted with a phenyl group.

As used herein, a “C₈-C₁₂ (phenyl)alkynyl” is a C₂-C₆ alkynyl group substituted with a phenyl group.

As used herein, a “C₁₁-C₁₆ (naphthyl)alkyl” is a C₁-C₆ alkyl group substituted with a naphthyl group.

As used herein, a “C₁₂-C₁₆ (naphthyl)alkenyl” is a C₂-C₆ alkenyl group substituted with a naphthyl group.

As used herein, a “C₁₂-C₁₆ (naphthyl)alkynyl” is a C₂-C₆ alkynyl group substituted with a naphthyl group.

As used herein, a “-3- to 9-membered heterocycle” is a 3- to 9-membered aromatic or nonaromatic monocyclic or bicyclic ring of carbon atoms and from 1 to 4 heteroatoms selected from oxygen, nitrogen and sulfur, and which can be substituted or unsubstituted. Examples of 3- to 9-membered heterocycles include, but are not limited to, aziridinyl, oxiranyl, thiiranyl, azirinyl, diaziridinyl, diazirinyl, oxaziridinyl, azetidinyl, azetidinonyl, oxetanyl, thietanyl, piperidinyl, piperazinyl, morpholinyl, pyrrolyl, oxazinyl, thiazinyl, diazinyl, triazinyl, tetrazinyl, imidazolyl, benzimidazolyl, tetrazolyl, indolyl, isoquinolinyl, quinolinyl, quinazolinyl, pyrrolidinyl, purinyl, isoxazolyl, benzisoxazolyl, furanyl, furazanyl, pyridinyl, oxazolyl, benzoxazolyl, thiazolyl, benzthiazolyl, thiophenyl, pyrazolyl, triazolyl, benzodiazolyl, benzotriazolyl, pyrimidinyl, isoindolyl and indazolyl.

A “5- to 9- membered ring” is a 5- to 9-membered aromatic or nonaromatic monocyclic or bicyclic ring of carbon atoms only, or of carbon atoms and from 1 to 4 heteroatoms selected from oxygen, nitrogen and sulfur, and which can be substituted or unsubstituted. In one embodiment, the 5- to 9-membered ring is a 5- to 7-membered monocyclic ring. In another embodiment, the 5- to 9-membered ring is a 7- to 9-membered bicyclic ring. Examples of 5- to 9-membered rings include, but are not limited to, cyclopentyl, cyclohexyl or cycloheptyl, which may be saturated or unsaturated, piperidinyl, piperazinyl, morpholinyl, pyrrolyl, oxazinyl, thiazinyl, diazinyl, triazinyl, tetrazinyl, imidazolyl, benzimidazolyl, tetrazolyl, indolyl, isoquinolinyl, quinolinyl, quinazolinyl, pyrrolidinyl, purinyl, isoxazolyl, benzisoxazolyl, furanyl, furazanyl, pyridinyl, oxazolyl, benzoxazolyl, thiazolyl, benzthiazolyl, thiophenyl, pyrazolyl, triazolyl, benzodiazolyl, benzotriazolyl, pyrimidinyl, isoindolyl and indazolyl.

As used herein, an —O-benzyl group can be substituted or unsubstituted.

As used herein, a -phenyl group can be substituted or unsubstituted.

When the groups described herein are said to be “substituted or unsubstituted,” when substituted, it is substituted with any desired substituent or substituents that maintains a Triheterocyclic Compound's usefulness for treating or preventing anemia or thrombocytopenia. Examples of substituents are those found in the illustrative compounds and embodiments disclosed herein, as well as halogen (chloro, iodo, bromo, or fluoro); C₁₋₆ alkyl; C₂₋₆ alkenyl; C₂₋₆ alkynyl; hydroxyl; C₁₋₆ alkoxyl; amino; nitro; thiol; thioether; imine; cyano; amido; phosphonato; phosphine; carboxyl; thiocarbonyl; sulfonyl; sulfonamide; ketone; aldehyde; ester; oxygen (═O); haloalkyl (e.g., trifluoromethyl); carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl); carbocyclic or heterocyclic, monocyclic or fused or non-fused polycyclic aryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl); benzyloxy; amino (primary, secondary, or tertiary); —N(CH₃)₂; O-lower alkyl; O-aryl, aryl; aryl-lower alkyl; CO₂CH₃; —OCH₂CH₃; methoxy; CONH₂; OCH₂CONH₂; NH₂; SO₂NH₂; OCHF₂; CF₃; OCF₃; and such moieties may also be optionally substituted by a fused-ring structure or bridge, for example —OCH₂O—.

Unless otherwise indicated, a substituent is unsubstituted.

These substituents may optionally be further substituted with a substituent selected from such groups.

In one embodiment, when administered to a subject, e.g., a mammal for veterinary use or a human for clinical use, the Triheterocyclic Compounds are administered in isolated form. As used herein, “isolated” means that the Triheterocyclic Compounds are separated from other components of either (a) a natural source, such as a plant or cell, in one embodiment, bacterial culture, or (b) a synthetic organic chemical reaction mixture. In another embodiment, via conventional techniques, the Triheterocyclic Compounds are purified. As used herein, “purified” means that when isolated, the isolate contains at least 95%, in one embodiment, at least 98%, of a single Triheterocyclic Compound by weight of the isolate.

It is recognized that Triheterocyclic Compounds of the invention can have one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding enantiomers and stereoisomers, that is, both the stereomerically pure form (e.g., geometrically pure, enantiomerically pure, or diastereomerically pure) and enantiomeric and stereoisomeric mixtures, e.g., racemates.

As used herein and unless otherwise indicated, the term “stereomerically pure” when used in connection with a chemical compound means a compound that comprises one stereoisomer of the compound and is substantially free of other stereoisomers of that compound. For example, a stereomerically pure compound having one chiral center is substantially free of the opposite enantiomer of the compound. A stereomerically pure compound having two chiral centers is substantially free of other diastereomers of the compound. In one embodiment, the stereomerically pure compound comprises greater than about 80% by weight of stereoisomer of the compound and less than about 20% by weight of other stereoisomers the compound, in another embodiment, greater than about 90% by weight of one stereoisomer of the compound and less than about 10% by weight of the other stereoisomers of the compound, and in still another embodiment, greater than about 95% by weight of one stereoisomer of the compound and less than about 5% by weight of the other stereoisomers of the compound, and in another embodiment, greater than about 97% by weight of one stereoisomer of the compound and less than about 3% by weight of the other stereoisomers of the compound.

Enantiomeric and stereoisomeric mixtures of compounds of the invention can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

### 5.2. Prodigiosin Compounds

As stated above, the present invention encompasses methods for treating or preventing anemia or thrombocytopenia, comprising administering an effective amount of a Prodigiosin Compound.

Illustrative Prodigiosin Compounds are disclosed, for example, in U.S. Patent Application Publication Nos. 2005-0014802, 2005-0267073 and 2006-0035945. Other illustrative Prodigiosin Compounds are discussed in U.S. Pat. Nos. 5,691,334 and 6,071,947. Still other illustrative Prodigiosin Compounds are disclosed in Furstner, A. (2003), Angew. Chem. Int. Ed. 42, 3582-3603; Manderville, R. (2001), Curr. Med. Chem. 1, 195; Rapoport and Holden, (1962) J. Am. Chem. Soc. 84, 653; Wasserman, H. et al. (2006), Bioorg. & Med. Chem. 16, 701; and d'Alessio, R. and Rossi, A. (1996) Synlett. 513. A “Prodigiosin Compound” is a free base or a pharmaceutically acceptable salt thereof.

In one embodiment, a “Prodigiosin Compound” is a compound of the formula: 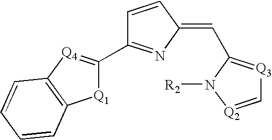
or the formula: 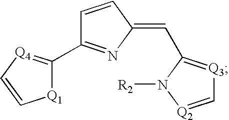
wherein Q₁-Q₄ and R₂ are as defined above for the compounds of formula (Ia), and each independently unsubstituted or further substituted as herein defined.

### 5.3. The Triheterocyclic Compounds of Formula (Ia)

As stated above, the present invention encompasses methods for treating or preventing anemia or thrombocytopenia, comprising administering a compound having the Formula (Ia) 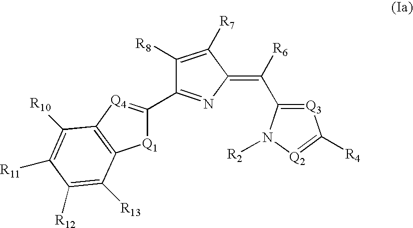
or a pharmaceutically acceptable salt thereof, wherein:

Q₁-Q₄, R₂, R₄, R₆—R₈ and R₁₀—R₁₃ are defined above for the compounds of formula (Ia).

A first subclass of the Triheterocyclic Compounds of Formula (Ia) is that wherein:

Q₁ is —NH—;

Q₂ is —C(R₃)—;

Q₃ is —C(R₅)—; and

Q₄ is —C(R₉)—.

A second subclass of the Triheterocyclic Compounds of Formula (Ia) is that wherein:

Q₁ is —O—;

Q₂ is —C(R₃)—;

Q₃ is —C(R₅)—; and

Q₄ is —C(R₉)—.

A third subclass of the Triheterocyclic Compounds of Formula (Ia) is that wherein:

Q₁ is —S—;

Q₂ is —C(R₃)—;

Q₃ is —C(R₅)—; and

Q₄ is —C(R₉)—.

A fourth subclass of the Triheterocyclic Compounds of Formula (Ia) is that wherein:

Q₁ is —NH—;

Q₂ is —N—;

Q₃ is —C(R₅)—; and

Q₄ is —C(R₉)—.

A fifth subclass of the Triheterocyclic Compounds of Formula (Ia) is that wherein:

Q₁ is —NH—;

Q₂ is —C(R₃)—;

Q₃ is —N—; and

Q₄ is —C(R₉)—.

A sixth subclass of the Triheterocyclic Compounds of Formula (Ia) is that wherein:

Q₁ is —NH—;

Q₂ is —C(R₃)—;

Q₃ is —C(R₅)—;

Q₄ is —CH—; and

R₂ and R₆ are —H.

A seventh subclass of the Triheterocyclic Compounds of Formula (Ia) is that wherein:

Q₁ is —NH—;

Q₂ is —C(R₃)—;

Q₃ is —C(R₅)—;

Q₄ is —CH—; and

R₂, R₄, R₆, R₈ and R₁₀—R₁₃ are —H.

An eighth subclass of the Triheterocyclic Compounds of Formula (Ia) is that wherein:

Q₁ is —NH—;

Q₂ is —C(C₁-C₈ alkyl)-;

Q₃ is —C(C₁-C₈ alkyl)-;

Q₄ is —CH—;

R₂, R₄, R₆, R₈ and R₁₀—R₁₃ are —H; and

R₇ is —O—(C₁-C₈ alkyl).

In certain specific embodiments, —O-benzyl is unsubstituted.

In certain specific embodiments, R₇ is 3-methoxy benzyloxy.

In certain specific embodiments, -phenyl is unsubstituted.

In certain specific embodiments, R₁₄ is (dimethylamino)phenyl. In even more specific embodiments, R₁ is C(O)NHR₁₄ and R₁₄ is (dimethylamino)phenyl.

In certain specific embodiments R₇ is —OCH₂C(O)OC₂H₅.

In certain specific embodiments, R₁₄ is (para-benzyloxy)phenyl. In even more specific embodiments, R₁ is C(O)NHR₁₄ and R₁₄ is (para-benzyloxy)phenyl.

In certain specific embodiments, R₁₄ is (para-bromo)phenyl. In even more specific embodiments, R₁ is —C(O)R₁₄ and R₁₄ is (para-bromo)phenyl.

In certain specific embodiments, Rₐ is (para-hydroxy)phenyl. In even more specific embodiments, Yₘ is —CH₂— and R₁₄ is (para-hydroxy)phenyl.

In certain specific embodiments, R₇ is —NH((para-methoxy)phenyl).

In certain specific embodiments R₁₁ is —(CH₂)₂OS(O)₂OH.

In certain specific embodiments, R₁₁ and R₁₂ are not joined together with the carbon atom to which each is attached.

In certain specific embodiments, R₆ is —H.

The present invention also provides compositions comprising a pharmaceutically acceptable carrier and an effective amount of a Triheterocyclic Compound of Formula (Ia).

An illustrative Triheterocyclic Compound of Formula (Ia) is:

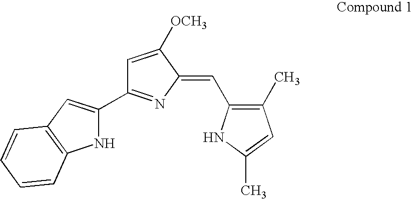

or a pharmaceutically acceptable salt thereof.

In one embodiment, Compound 1's pharmaceutically acceptable salt is a tartrate salt.

In another embodiment, Compound 1's pharmaceutically acceptable salt is a mesylate salt.

In one embodiment, the invention provides a composition comprising a pharmaceutically acceptable carrier and Compound 1 or a pharmaceutically acceptable salt thereof. In another embodiment, the pharmaceutically acceptable salt is a tartrate salt. In even another embodiment, the pharmaceutically acceptable salt is a mesylate salt.

Other illustrative Triheterocyclic Compounds of Formula (Ia) are shown below: 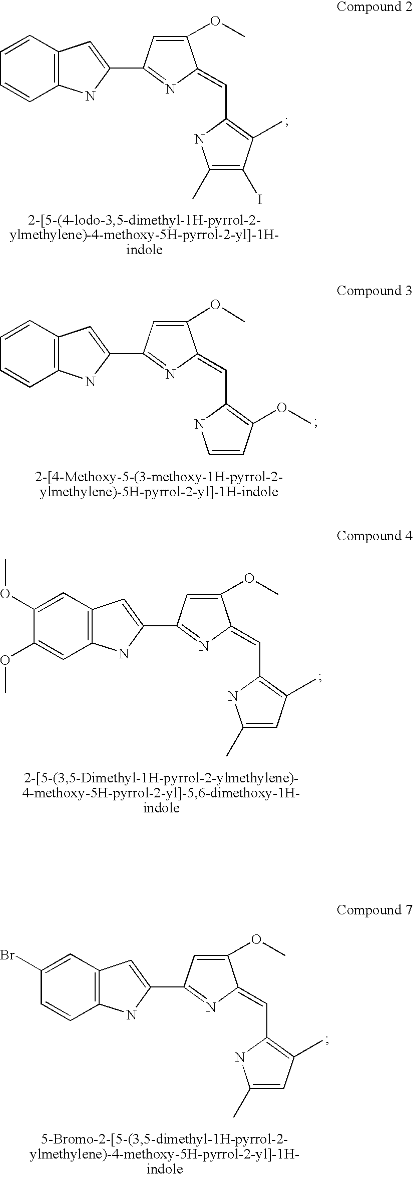 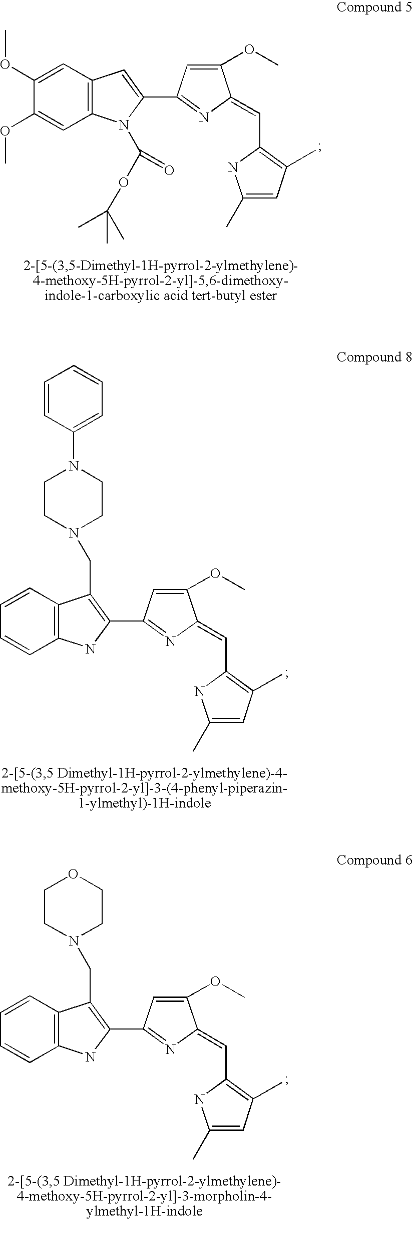 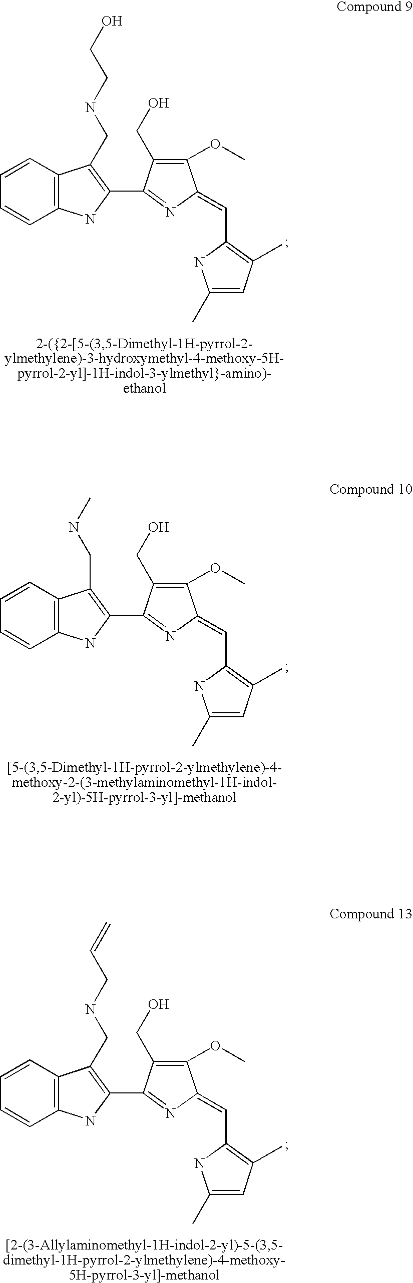 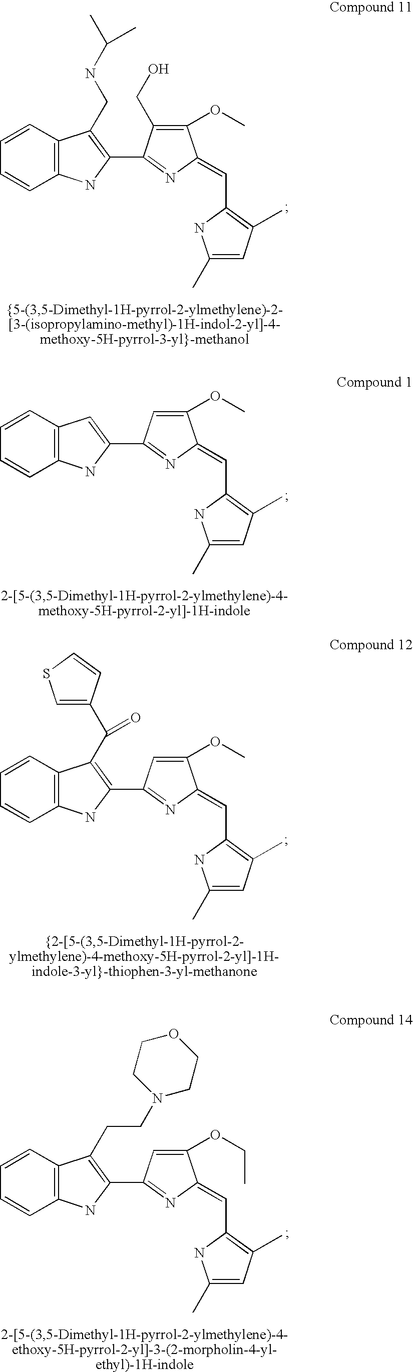 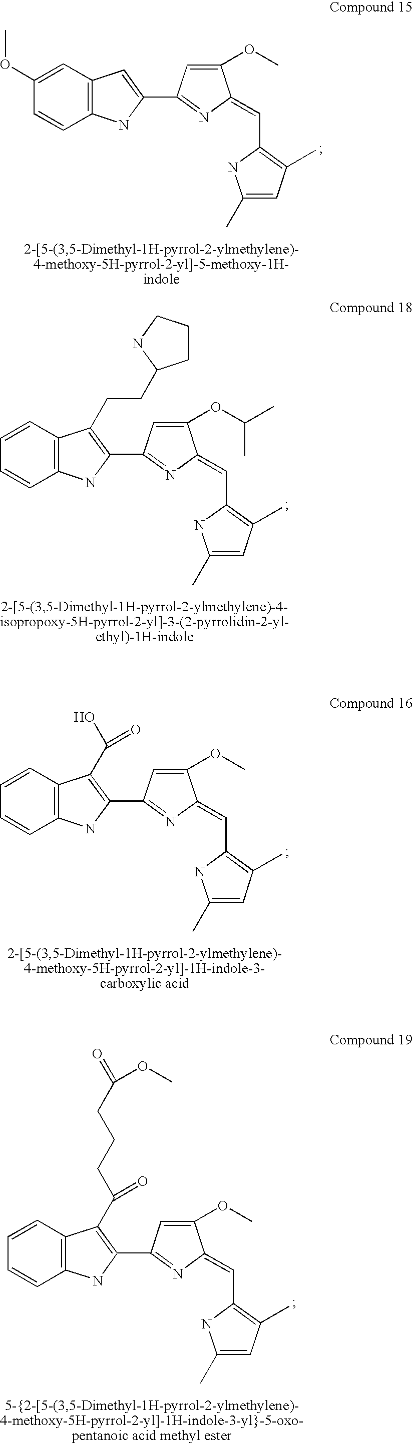 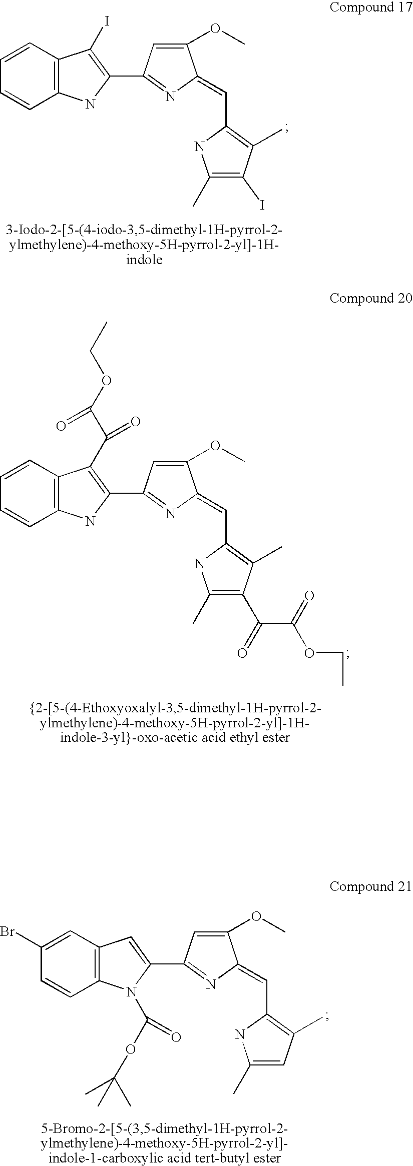 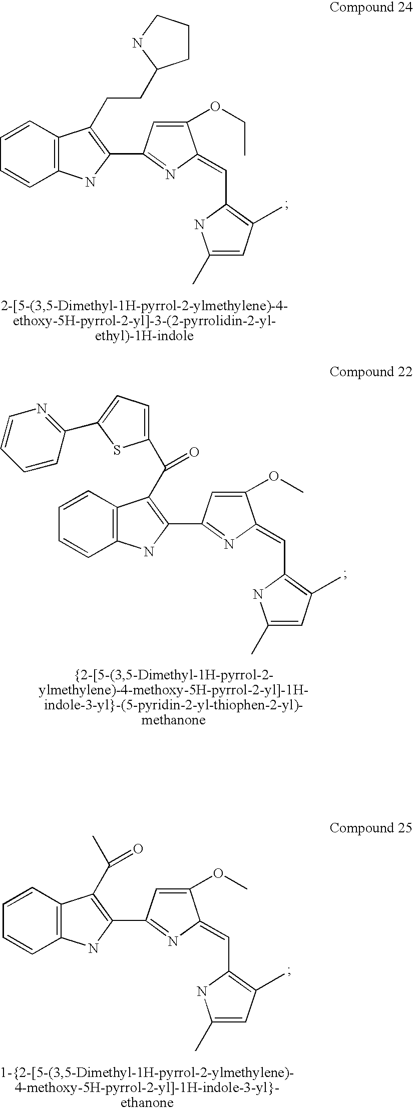 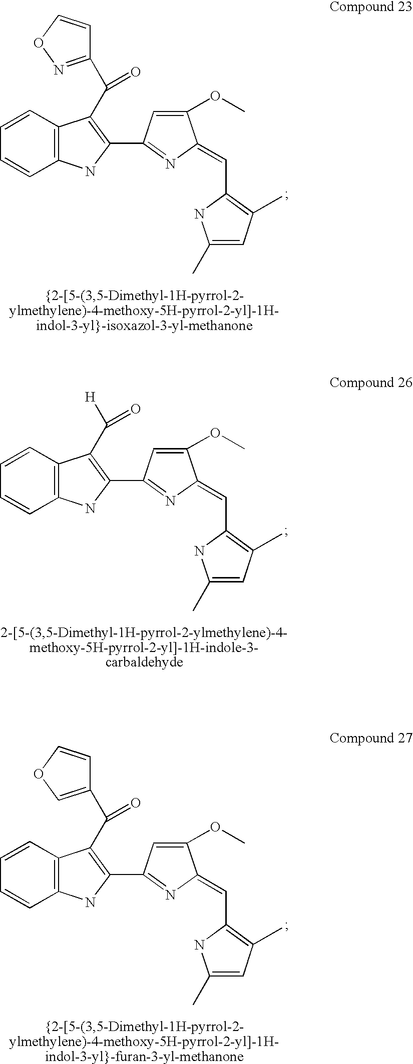 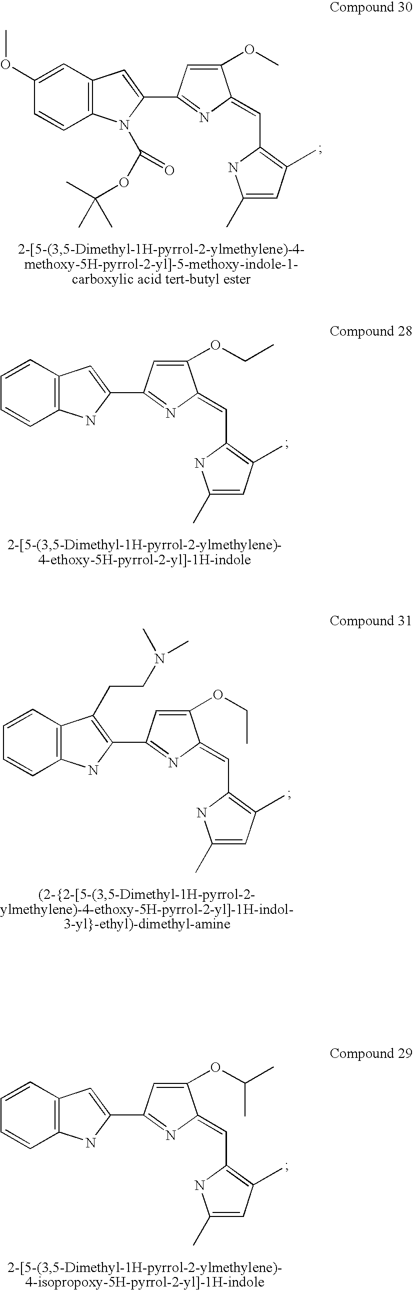 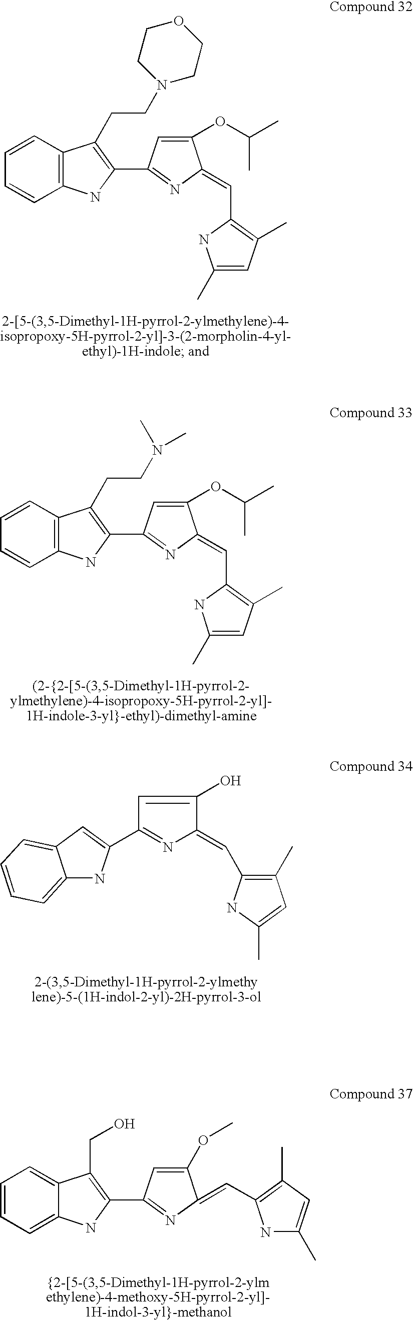 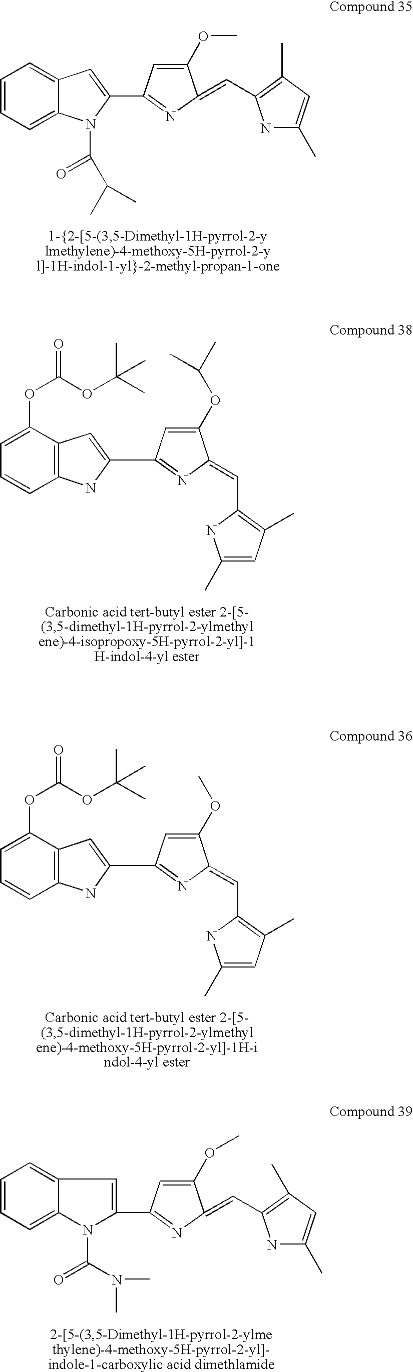 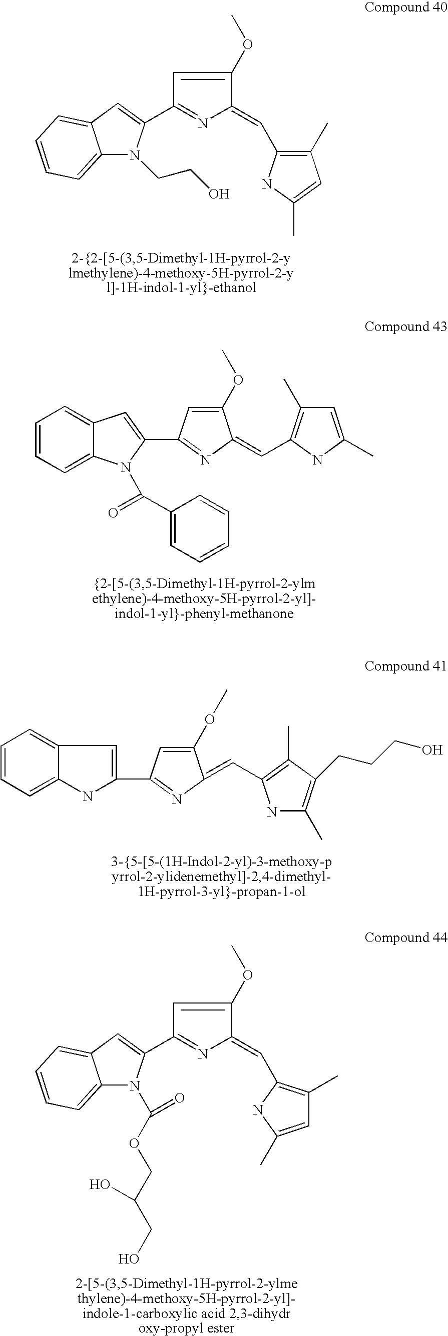 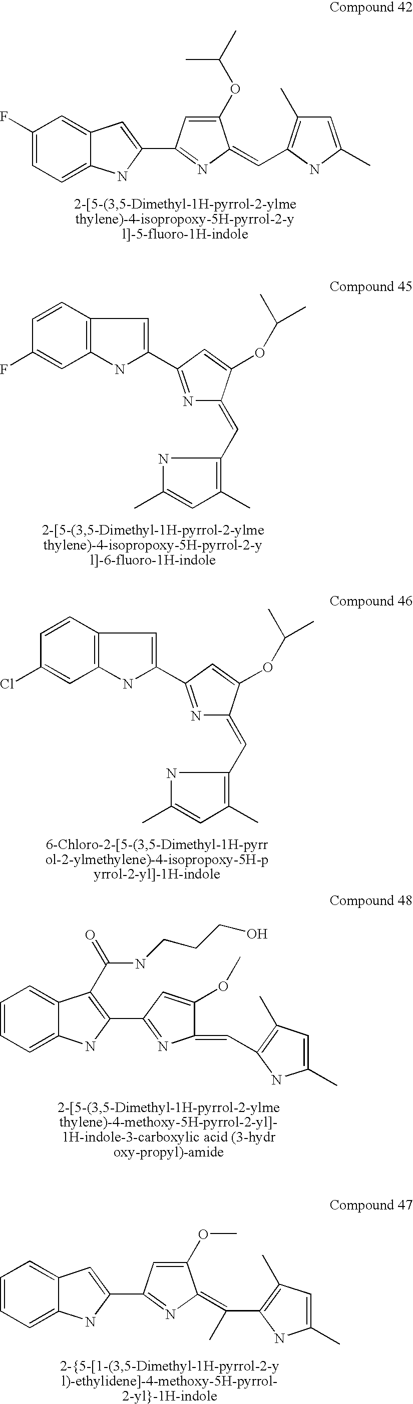 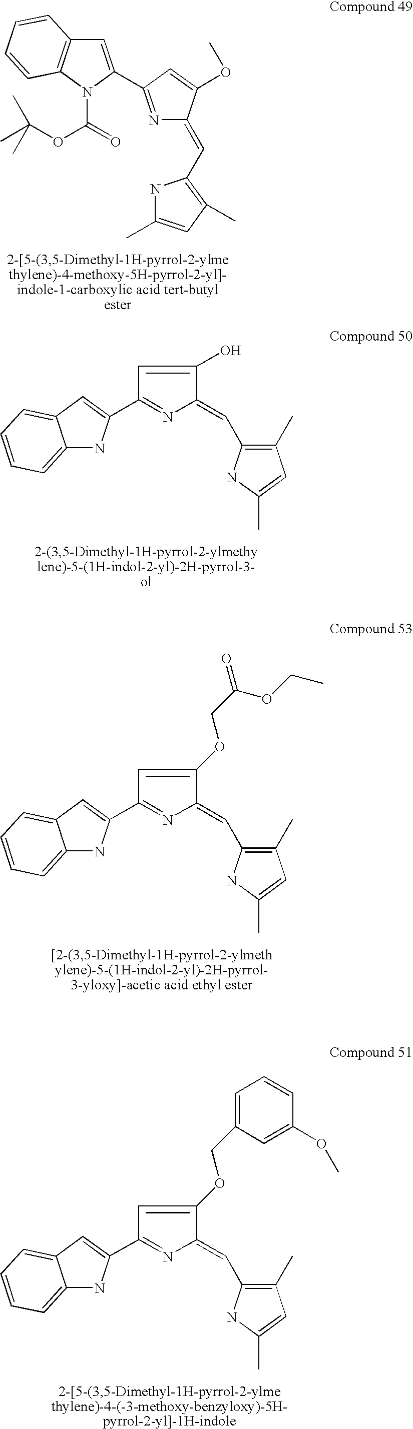 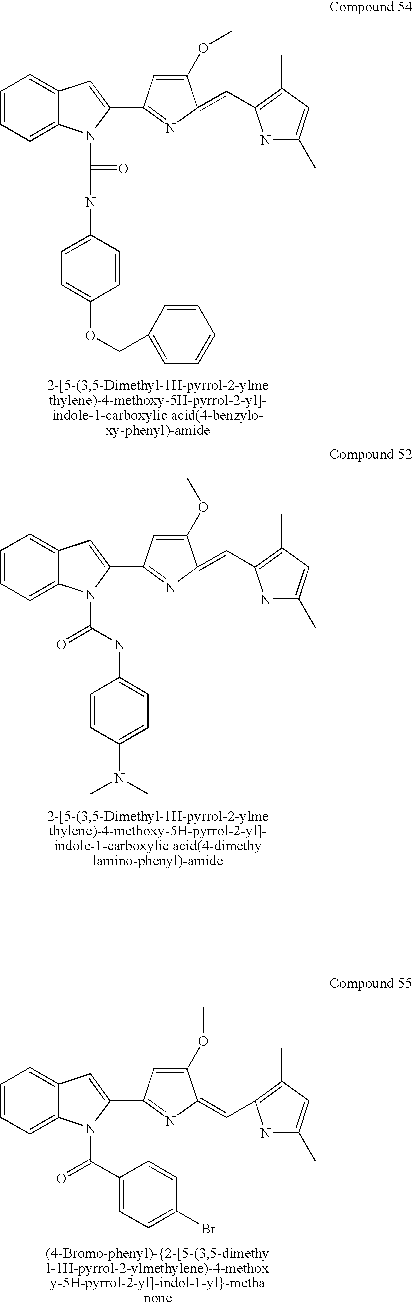 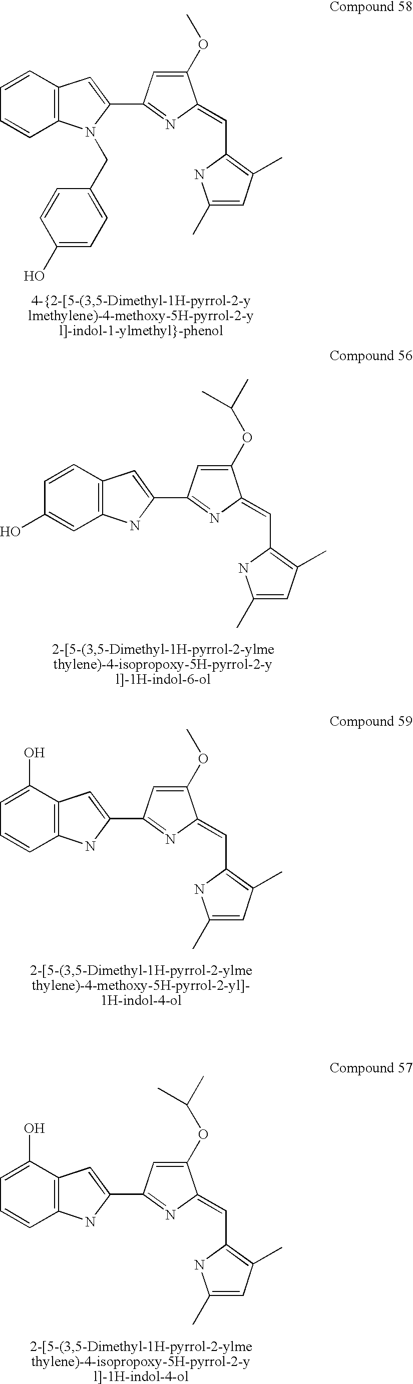 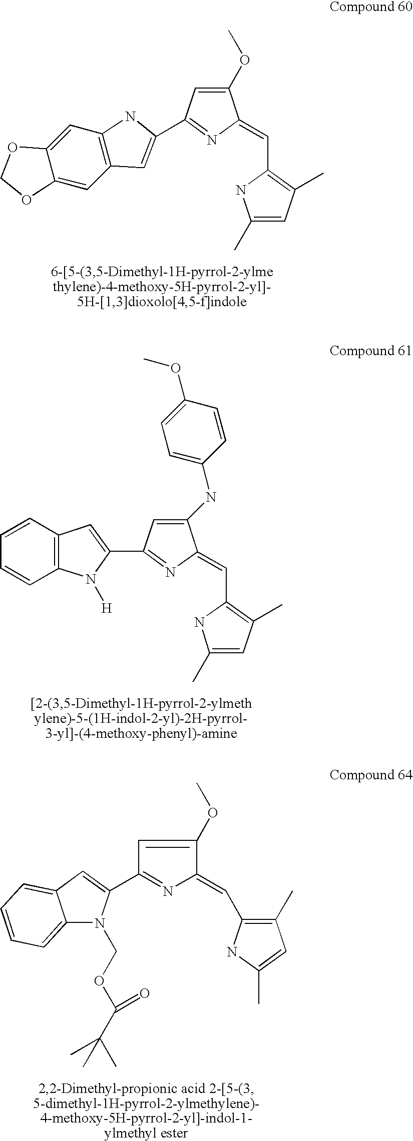 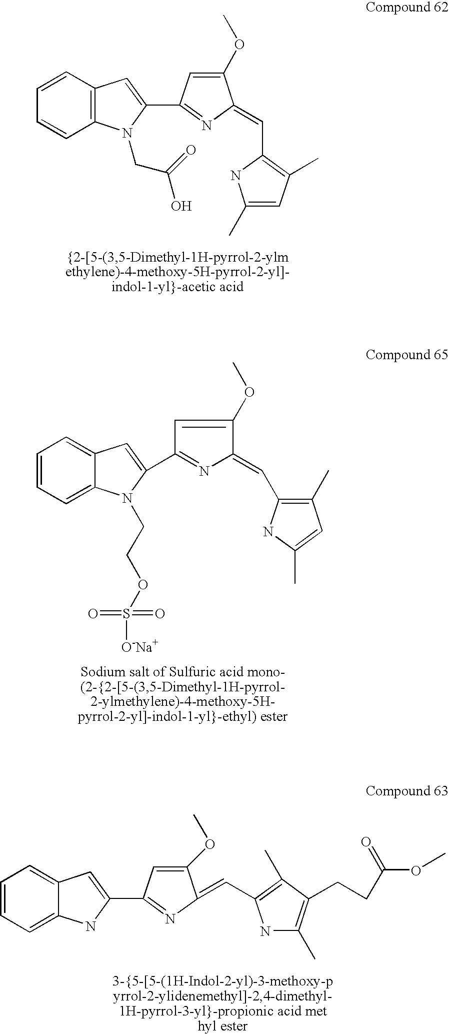
and pharmaceutically acceptable salts thereof.

### 5.4. The Triheterocyclin Compounds of Formula (Ib)

As stated above, the present invention encompasses methods for treating or preventing anemia or thrombocytopenia, comprising administering a compound having the Formula (Ib) 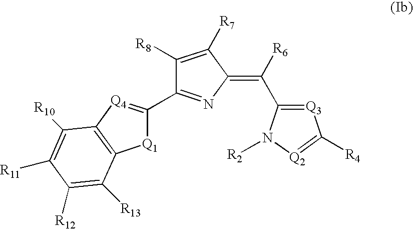
or a pharmaceutically acceptable salt thereof, wherein:

Q₁-Q₄, R₂, R₄, R₆—R₈ and R₁₀—R₁₃ are defined above for the compounds of formula (Ib).

A first subclass of the Triheterocyclic Compounds of Formula (Ib) is that wherein:

Q₁ is —NH—;

Q₂ is —C(R₃)—;

Q₃ is —C(R₅)—; and

Q₄ is —C(R₉)—.

A second subclass of the Triheterocyclic Compounds of Formula (Ib) is that wherein:

Q₁ is —O—;

Q₂ is —C(R₃)—;

Q₃ is —C(R₅)—; and

Q₄ is —C(R₉)—.

A third subclass of the Triheterocyclic Compounds of Formula (Ib) is that wherein:

Q₁ is —S—;

Q₂ is —C(R₃)—;

Q₃ is —C(R₅)—; and

Q₄ is —C(R₉)—.

A fourth subclass of the Triheterocyclic Compounds of Formula (Ib) is that wherein:

Q₁ is —NH—;

Q₂is —N—;

Q₃ is —C(R₅)—; and

Q₄ is —C(R₉)—.

A fifth subclass of the Triheterocyclic Compounds of Formula (Ib) is that wherein:

Q₁ is —NH—;

Q₂ is —C(R₃)—;

Q₃ is —N—; and

Q₄ is —C(R₉)—.

A sixth subclass of the Triheterocyclic Compounds of Formula (Ib) is that wherein:

Q₁ is —NH—;

Q₂ is —C(R₃)—;

Q₃ is —C(R₅)—;

Q₄ is —CH—; and

R₂ and R₆ are —H.

A seventh subclass of the Triheterocyclic Compounds of Formula (Ib) is that wherein:

Q₁ is —NH—;

Q₂ is —C(R₃)—;

Q₃ is —C(R₅)—;

Q₄ is —CH—; and

R₂, R₄, R₆, R₈ and R₁₀—R₁₃ are —H.

An eighth subclass of the Triheterocyclic Compounds of Formula (Ib) is that wherein:

Q₁ is —NH—;

Q₂ is —C(C₁-C₈ alkyl)-;

Q₃ is —C(C₁-C₈ alkyl)-;

Q₄ is —CH—;

R₂, R₄, R₆, R₈ and R₁₀—R₁₃ are —H; and

R₇ is —O—(C₁-C₈ alkyl).

In certain specific embodiments, —O-benzyl is unsubstituted.

In certain specific embodiments, R₇ is 3-methoxy benzyloxy.

In certain specific embodiments, -phenyl is unsubstituted.

In certain specific embodiments, R₁₄ is (dimethylamino)phenyl. In even more specific embodiments, R₁ is C(O)NHR₁₄ and R₁₄ is (dimethylamino)phenyl.

In certain specific embodiments R₇ is —OCH₂C(O)OC₂H₅.

In certain specific embodiments, R₁₄ is (para-benzyloxy)phenyl. In even more specific embodiments, R₁ is C(O)NHR₁₄ and R₁₄ is (para-benzyloxy)phenyl.

In certain specific embodiments, R₁₄ is (para-bromo)phenyl. In even more specific embodiments, R₁ is —C(O)R₁₄ and R₁₄ is (para-bromo)phenyl.

In certain specific embodiments, Rₐ is (para-hydroxy)phenyl. In even more specific embodiments, Yₘ is —CH₂— and R₁₄ is (para-hydroxy)phenyl.

In certain specific embodiments, R₇ is —NH((para-methoxy)phenyl).

In certain specific embodiments R₁ is —(CH₂)₂OS(O)₂OH.

In certain specific embodiments, R₁₁ and R₁₂ are not joined together with the carbon atom to which each is attached.

The present invention also provides compositions comprising a pharmaceutically acceptable carrier and an effective amount of a Triheterocyclic Compound of Formula (Ib).

### 5.5. The Triheterocyclic Compounds of Formula (II)

As stated above, the present invention encompasses methods for treating or preventing anemia or thrombocytopenia, comprising administering a compound having the Formula (II) 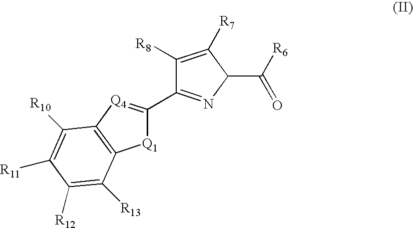
or a pharmaceutically acceptable salt thereof, wherein: Q₁, Q₄, R₆—R₈ and R₁₀—R₁₃ are defined above for the compounds of Formula (II).

A first subclass of the Triheterocyclic Compounds of Formula (II) is that wherein:

Q₁ is —NH—; and

Q₄ is —C(R₉)—.

A second subclass of the Triheterocyclic Compounds of Formula (II) is that wherein:

Q₁ is —O—; and

Q₄ is —C(R₉)—.

A third subclass of the Triheterocyclic Compounds of Formula (II) is that wherein:

Q₁ is —S—; and

Q₄ is —C(R₉)—.

A fourth subclass of the Triheterocyclic Compounds of Formula (II) is that wherein:

Q₁ is —NH—;

Q₄ is —CH—; and

R₆ is —H.

A fifth subclass of the Triheterocyclic Compounds of Formula (II) is that wherein:

Q₁ is —NH—;

Q₄ is —CH—;

R₆ is —H; and

R₁₀—R₁₃ are —H.

A sixth subclass of the Triheterocyclic Compounds of Formula (II) is that wherein:

Q₁ is —NH—;

Q₄ is —CH—;

R₆ is —H;

R₈ and R₁₀—R₁₃ are —H; and

R₇ is —O—(C₁-C₈ alkyl).

The present invention also provides compositions comprising a pharmaceutically acceptable carrier and an effective amount of a compound of Formula (II).

### 5.6. The Triheterocyclic Compounds of Formula (Ic)

As stated above, the present invention encompasses methods for treating or preventing anemia or thrombocytopenia, comprising administering a compound having the Formula (Ic):

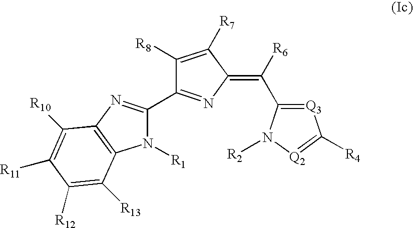

or a pharmaceutically acceptable salt thereof, wherein: Q₁, Q₄, R₆—R₈ and R₁₀—R₁₃ are defined above for the compounds of Formula (Ic).

In certain specific embodiments, —O-benzyl is unsubstituted.

In certain specific embodiments, R₇ is 3-methoxy benzyloxy.

In certain specific embodiments, -phenyl is unsubstituted.

In certain specific embodiments, R₁₄ is (dimethylamino)phenyl. In even more specific embodiments, R₁ is C(O)NHR₁₄ and R₁₄ is (dimethylamino)phenyl.

In certain specific embodiments R₇ is —OCH₂C(O)OC₂H₅.

In certain specific embodiments, R₁₄ is (para-benzyloxy)phenyl. In even more specific embodiments, R₁ is C(O)NHR₁₄ and R₁₄ is (para-benzyloxy)phenyl.

In certain specific embodiments, R₁₄ is (para-bromo)phenyl. In even more specific embodiments, R₁ is —C(O)R₁₄ and R₁₄ is (para-bromo)phenyl.

In certain specific embodiments, Rₐ is (para-hydroxy)phenyl. In even more specific embodiments, Yₘ is —CH₂— and R₁₄ is (para-hydroxy)phenyl.

In certain specific embodiments, R₇ is —NH((para-methoxy)phenyl).

In certain specific embodiments R₁ is —(CH₂)₂OS(O)₂OH.

In certain specific embodiments, R₁₁ and R₁₂ are not joined together with the carbon atom to which each is attached.

The present invention also provides compositions comprising a pharmaceutically acceptable carrier and an effective amount of a compound of formula (Ic).

### 5.7. Prodrugs of Triheterocyclic Compounds

The present invention also encompasses methods for treating or preventing anemia or thrombocytopenia, comprising administering a prodrug of a Triheterocyclic Compound of the invention. Prodrugs include derivatives of Triheterocyclic Compounds that can hydrolyze, oxidize, or otherwise react under biological conditions (in vitro or in vivo) to provide an active Triheterocyclic Compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable sulfate, and biohydrolyzable phosphate analogues. In certain embodiments, prodrugs of Triheterocyclic Compounds with carboxyl functional groups are the lower alkyl esters of the carboxylic acid. The carboxylate esters are conveniently formed by esterifying any of the carboxylic acid moieties present on the molecule. In one embodiment, the biohydrolyzable moiety is present on a ring nitrogen of the Triheterocyclic Compound.

Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh). Biohydrolyzable moieties of a Triheterocyclic Compounds 1) do not interfere with the biological activity of the compound but can confer upon that compound advantageous properties in vivo, such as uptake, duration of action, or onset of action; or 2) are biologically inactive but are converted in vivo to the biologically active compound. Examples of biohydrolyzable esters include, but are not limited to, lower alkyl esters, alkoxyacyloxy esters, alkyl acylamino alkyl esters, and choline esters. Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

In one embodiment, the present invention encompasses methods for treating or preventing anemia or thrombocytopenia, comprising administering a prodrug of the Triheterocyclic Compound, which is either Compound 66 or 67, or both: 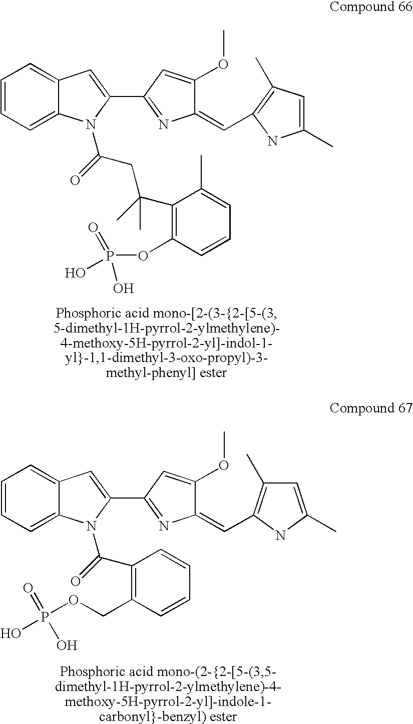
or a pharmaceutically acceptable salt thereof.

Illustrative methods for synthesizing Compound 66 or Compound 67 are described in U.S. Patent Application Publication No. 2005-0267073.

The present invention also provides compositions comprising a pharmaceutically acceptable carrier and an effective amount of a prodrug of a Triheterocyclic Compound.

### 5.8. Methods for Making the Triheterocyclic Compounds

The Triheterocyclic Compounds can be obtained via standard, well-known synthetic methodology, see, e.g., March, J. Advanced Organic Chemistry: Reactions, Mechanisms, and Structure, 4^{th} ed., 1992. Illustrative methods for preparing the Triheterocyclic Compounds are described, for example, in U.S. Pat. Nos. 5,691,334 and 6,071,947; and U.S. Patent Application Publication Nos. 2005-0014802, 2006-0035945, and 2005-0267073. Starting materials useful for preparing the Triheterocyclic Compounds are commercially available or can be prepared from commercially available materials using known synthetic methods and reagents.

### 5.9. Therapeutic/Prophylactic Administration and Compositions

Due to their activity, the Triheterocyclic Compounds are advantageously useful in veterinary and human medicine. For example, the Triheterocyclic Compounds are useful for the treatment or prevention of anemia or thrombocytopenia.

The invention provides methods of treatment and prophylaxis by administration to a subject of an effective amount of a Triheterocyclic Compound. The subject can be an animal, including, but not limited, a human, mammal, or non-human animal such as a cow, horse, sheep, pig, chicken, turkey, quail, cat, dog, mouse, rat, rabbit, mouse or guinea pig, and is in one embodiment a mammal, such as a human.

The present compositions, which comprise an effective amount of a Triheterocyclic Compound, can be administered by any convenient route, for example by infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer a Triheterocyclic Compound. In certain embodiments, more than one Triheterocyclic Compound is administered to a subject. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically to the ears, nose, eyes, or skin. The mode of administration can be left to the discretion of the practitioner, and can depend in part upon the subject's condition and overall treatment regime.

Pulmonary administration can also be employed, e.g., by use of an inhaler or nebulizer, and formulating with an aerosolizing agent, or via perfusion in a fluorocarbon or synthetic pulmonary surfactant. In certain embodiments, the Triheterocyclic Compounds can be formulated as a suppository, with traditional binders and carriers such as triglycerides.

In another embodiment, the Triheterocyclic Compounds can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid.)

In yet another embodiment, the Triheterocyclic Compounds can be delivered in a controlled-release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled-release system can be placed in proximity of the target of the Triheterocyclic Compounds, e.g., the brain, thus requiring only a fraction of the systemic dose (see, e.g., Goodson, in Medical Applications of Controlled Release, supra, vol. 2, pp. 115-138 (1984)). Other controlled-release systems discussed in the review by Langer (Science 249:1527-1533 (1990)) may be used.

The present compositions comprise an effective amount of a Triheterocyclic Compound and a pharmaceutically acceptable carrier. In certain embodiments, the present compositions further comprise another therapeutic agent.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which a Triheterocyclic Compound is administered. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. In addition, auxiliary, stabilizing, thickening, lubricating and coloring agents may be used. When administered to a subject, the Triheterocyclic Compounds and pharmaceutically acceptable carriers can be sterile. In one embodiment, water is a carrier when the Triheterocyclic Compound is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, can also contain minor amounds of wetting or emulsifying agents, or pH buffering agents.

The present compositions can take the form of solutions, suspensions, emulsion, tablets, pills, pellets, capsules, capsules containing liquids, powders, sustained-release Formulations, suppositories, emulsions, aerosols, sprays, suspensions, or any other form suitable for use. In one embodiment, the pharmaceutically acceptable carrier is a capsule (see e.g., U.S. Pat. No. 5,698,155). Other examples of suitable pharmaceutical carriers are described in “Remmington's Pharmaceutical Sciences” by E. W. Martin.

In another embodiment, the Triheterocyclic Compounds are formulated in accordance with routine procedures as a pharmaceutical composition adapted for intravenous administration to human beings. Typically, Triheterocyclic Compounds for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the Triheterocyclic Compound is to be administered by infusion, it can be dispensed, for example, with an infusion bottle containing sterile pharmaceutical grade water or saline. Where the Triheterocyclic Compound is administered by injection, an ampoule of sterile water for injection or saline can be provided so that the ingredients may be mixed prior to administration.

Compositions for oral delivery may be in the form of tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered Triheterocyclic Compounds. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate may also be used. Oral compositions can include standard carriers such as mannitol, lactose, starch, magnesium stearate, sodium saccharine, cellulose, or magnesium carbonate. Such carriers can be of pharmaceutical grade.

The amount of the Triheterocyclic Compound that is effective for the treatment or prevention of a particular disorder or condition can depend on the nature of the disorder or condition, and can be determined by standard clinical techniques. In addition, in vitro or in vivo assays may optionally be employed to help identify optimal dosage ranges. The precise dose to be employed in the compositions can also depend on the route of administration, and the seriousness of the disease or disorder, and can be decided according to the judgment of the practitioner and each subject's circumstances and stage of disease.

Suitable effective dosage ranges for intravenous administration are generally about 3.0 to 90 mg, in another embodiment, about 3.5 to 80 mg of Triheterocyclic Compound per square meter of body surface area. In specific embodiments, the i.v. dose is about 3.5 to about 5.0 mg/m², about 5.0 to about 10 mg/m², about 10.0 to about 20 mg/m², about 20 to about 28 mg/m², about 28 to about 40 mg/m², about 40 to about 50 mg/rn², about 50 to about 65 mg/m², about 65 to 72 mg/m², or about 72 mg/m² to about 80 mg/m² (or the equivalent doses expressed per kilogram body weight). In specific embodiments, the i.v. dose is about 3.0 mg/m², about 3.5 mg/m², about 4.0 mg/m², about 4.5 mg/m², about 5.0 mg/m², about 5.5 mg/m², about 6.0 mg/m², about 6.5 mg/m², about 7.0 mg/m², about 7.5 mg/m², about 8.0 mg/m², about 8.5 mg/m², about 9.0 mg/m², about 9.5 mg/m², about 10 mg/m², about 11 mg/m², about 12 mg/m², about 13 mg/m², about 14 mg/m², about 15 mg/m², about 16 mg/m², about 17 mg/m², about 18 mg/m², about 19 mg/m², about 20 mg/m², about 21 mg/m², about 22 mg/m², about 23 mg/m², about 24 mg/m², about 25 mg/m², about 26 mg/m², about 27 mg/m², about 28 mg/m², about 29 mg/m², about 30 mg/m², about 31 mg/m², about 32 mg/m², about 33 mg/m², about 34 mg/m², about 35 mg/m², about 36 mg/m², about 37 mg/m², about 38 mg/m², about 39 mg/m², about 40 mg/m², about 41 mg/m², about 42 mg/m², about 43 mg/m², about 44 mg/m², about 45 mg/m², about 46 mg/m², about 47 mg/m², about 48 mg/m², about 49 mg/m², about 50 mg/m², about 51 mg/m², about 52 mg/m², about 53 mg/m², about 54 mg/m², about 55 mg/m², about 56 mg/m², about 57 mg/m², about 58 mg/m², about 59 mg/m², about 60 mg/m², about 61 mg/m², about 62 mg/m², about 63 mg/m², about 64 mg/m², about 65 mg/m², about 66 mg/m², about 67 mg/m², about 68 mg/m², about 69 mg/m², about 70 mg/m², about 71 mg/m², about 72 mg/m², about 73 mg/m², about 74 mg/m², about 75 mg/m², about 76 mg/m², about 77 mg/m2 , about 78 mg/m², about 79 mg/m², about 80 mg/m², about 81 mg/m², about 82 mg/m², about 83 mg/m², about 84 mg/m², about 85 mg/m², about 86 mg/m², about 87 mg/m², about 88 mg/m², about 89 mg/m², or about 90 mg/m² (or the equivalent doses expressed per kilogram body weight). Alternatively, a suitable dose range for i.v. administration is about 3.5 to 80 mg, without adjustment for a subject's body weight or body surface area.

Suitable dosage ranges for intranasal administration are generally about 0.01 pg/kg body weight to 1 mg/kg body weight. Suppositories generally contain 0.5% to 10% by weight of one or more Triheterocyclic Compounds alone or in combination with another therapeutic agent. A composition can contain about 10% to about 95% by weight of one or more Triheterocyclic Compounds alone or in combination with another therapeutic agent.

In specific embodiments of the invention, suitable dose ranges for oral administration are generally about 3.0 to about 90 mg, in another embodiment, about 3.5 to about 10.0 mg, and in another embodiment, about 10 to about 40 mg of Triheterocyclic Compound per square meter of body surface area or their equivalent doses expressed per kilogram body weight. In specific embodiments the oral dose is about 3.0 to about 5.0 mg/m², about 5.0 to 10 mg/m², about 10 to about 20 mg/m², about 20 to about 28 mg/m², about 28 to 40 mg/m², about 40 to about 50 mg/m², about 50 to about 60 mg/m², about 60 mg/m² to about 70 mg/m², about 70 mg/m² to about 80 mg/m² (or the equivalent doses expressed per kilogram body weight). In specific embodiments, the oral dose is about 3.0 mg/m², about 3.5 mg/m², about 4.0 mg/m², about 4.5 mg/m², about 5.0 mg/m², about 5.5 mg/m², about 6.0 mg/m², about 6.5 mg/m², about 7.0 mg/m², about 7.5 mg/m², about 8.0 mg/m², about 8.5 mg/m², about 9.0 mg/m², about 9.5 mg/m², about 10 mg/m², about 11 mg/m², about 12 mg/m², about 13 mg/m², about 14 mg/m², about 15 mg/m², about 16 mg/m², about 17 mg/m², about 18 mg/m², about 19 mg/m², about 20 mg/m², about 21 mg/m², about 22 mg/m², about 23 mg/m², about 24 mg/m², about 25 mg/m², about 26 mg/m², about 27 mg/m², about 28 mg/m², about 29 mg/m², about 30 mg/m , about 31 mg/m², about 32 mg/m², about 33 mg/m², about 34 mg/m², about 35 mg/m², about 36 mg/m², about 37 mg/m², about 38 mg/m², about 39 mg/m², about 40 mg/m², about 41 mg/m², about 42 mg/m², about 43 mg/m², about 44 mg/m², about 45 mg/m², about 46 mg/m², about 47 mg/m², about 48 mg/m², about 49 mg/m², about 50 mg/m², about 51 mg/m², about 52 mg/m², about 53 mg/m², about 54 mg/m², about 55 mg/m², about 56 mg/m², about 57 mg/m², about 58 mg/m², about 59 mg/m², about 60 mg/m², about 61 mg/m², about 62 mg/m², about 63 mg/m², about 64 mg/m², about 65 mg/m², about 66 mg/m², about 67 mg/m², about 68 mg/m², about 69 mg/m², about 70 mg/m², about 71 mg/m², about 72 mg/m², about 73 mg/m², about 74 mg/m², about 75 mg/m², about 76 mg/m², about 77 mg/m², about 78 mg/m², about 79 mg/m², about 80 mg/m², about 81 mg/m², about 82 mg/m², about 83 mg/m², about 84 mg/m², about 85 mg/m², about 86 mg/m², about 87 mg/m², about 88 mg/m², about 89 mg/m², or about 90 mg/m² (or the equivalent doses expressed per kilogram body weight). In another embodiment, a suitable dose range for oral administration is from about 3.5 to 80 mg, without adjustment for a subject's body weight or body surface area. Other effective doses may be extrapolated from dose-response curves derived from in vitro or animal model test systems. Such animal models and systems are known in the art.

The invention also provides pharmaceutical packs or kits comprising one or more containers containing one or more Triheterocyclic Compounds. Optionally associated with such container(s) can be a notice in the form prescribed by a governmental agency regulating the manufacture, use or sale of pharmaceuticals or biological products, which notice reflects approval by the agency of manufacture, use or sale for human administration. In certain embodiments, e.g., when administered for the treatment or prevention of anemia or thrombocytopenia, the kit may also contain one or more therapeutic agents useful for treating the underlying disease to be administered in combination with a Triheterocyclic Compound.

The Triheterocyclic Compounds can be assayed in vitro, and then in vivo, for the desired therapeutic or prophylactic activity, prior to use in humans.

In one embodiment, a subject tissue sample is grown in culture, and contacted or otherwise administered with a Triheterocyclic Compound, and the effect of such Triheterocyclic Compound upon the tissue sample is observed and compared to a non-contacted tissue. In other embodiments, a cell culture model is used in which the cells of the cell culture are contacted or otherwise administered with a Triheterocyclic Compound, and the effect of such Triheterocyclic Compound upon the tissue sample is observed and compared to a non-contacted cell culture. Such Triheterocyclic Compounds may also be demonstrated effective and safe using animal model systems developed for various forms of anemia or thrombocytopenia. Specifically, U.S. Pat. No. 6,956,022 teaches methods of testing the compound to treat thrombocytopenia in mice. Other examples of known animal models include one for the study of sickle cell anemia (Xianfeng, L. et al. (1997), Blood, 90: 4620-4627); alloimmune thrombocyptopenia (Mylvaganam, R et al. (1984), Clin. Immunol. Immunopathol., 2: 163-170; and heparin-induced thrombocytopenia (Reilly et al. (2001), Blood, 98: 2442-2447.

Other methods will be known to the skilled artisan and are within the scope of the invention.

### 5.10. Treatment or Prevention of Anemia

In certain embodiments, the Triheterocyclic Compound can be used in the treatment or prevention of anemia, such as, but not limited to the following:

Drug-induced anemia (Non-limiting examples of anemia-inducing drugs include: Penicillin and its derivatives, Cephalosporins, Levodopa, Methyldopa, Quinidine, anti-inflammatory drugs, and chemotherapeutic or anti-viral agents (e.g. AZT)); radiation-induced anemia; anemia associated with relative erythropoietin deficiency; anemias of chronic diseases (ACD); anemia associated with kidney diseases, or splenomegaly; aplastic anemia, anemia associated with bone marrow disease; hemolytic anemias (autoimmune disorders, chemical or toxin, diseases of the liver); cancer-induced anemia, where the cancer is such as, but not limited to, breast, prostate, lung, skin, colorectal, cervical, pancreatic, ovarian, lymphoma, leukemia, liver, testicular, and brain cancer; and defective hemoglobin (Sickle cell anemia, Thalassemia).

In certain embodiments, the Triheterocyclic Compound can be used in the treatment or prevention of anemia induced by one or more of the cancers or neoplasic disorders listed in Table 1:

TABLE 1
CANCERS AND NEOPLASTIC DISORDERS

```
Leukemia
  acute leukemia
  acute t-cell leukemia
  acute lymphocytic leukemia
  acute myelocytic leukemia
    myeloblastic
    promyelocytic
    myelomonocytic
    Monocytic
  erythroleukemia
  chronic leukemia
  chronic myelocytic (granulocytic) leukemia
  chronic lymphocytic leukemia
  myelodysplastic syndromes (MDS)
Polycythemia vera
Lymphoma
  Hodgkin's disease
  non-Hodgkin's disease
Multiple myeloma
Waldenström's macroglobulinemia
Heavy chain disease
Solid tumors
  sarcomas and carcinomas
    fibrosarcoma
    myxosarcoma
    liposarcoma
    chondrosarcoma
    osteogenic sarcoma
    chordoma
    angiosarcoma
    endotheliosarcoma
    lymphangiosarcoma
    lymphangioendotheliosarcoma
    synovioma
    mesothelioma
    Ewing's tumor
    leiomyosarcoma
    rhabdomyosarcoma
    colon carcinoma
    pancreatic cancer
    breast cancer
    ovarian cancer
    prostate cancer
    squamous cell carcinoma
    basal cell carcinoma
    adenocarcinoma
    sweat gland carcinoma
    sebaceous gland carcinoma
    papillary carcinoma
    papillary adenocarcinomas
    cystadenocarcinoma
    medullary carcinoma
    bronchogenic carcinoma
    renal cell carcinoma
    hepatoma
    bile duct carcinoma
    choriocarcinoma
    seminoma
    embryonal carcinoma
    Wilms' tumor
    cervical cancer
    uterine cancer
    testicular tumor
    lung carcinoma
    small cell lung carcinoma
    bladder carcinoma
    epithelial carcinoma
    glioma
    astrocytoma
    medulloblastoma
    craniopharyngioma
    ependymoma
    pinealoma
    hemangioblastoma
    acoustic neuroma
    oligodendroglioma
    meningioma
    melanoma
    neuroblastoma
    retinoblastoma
```

In certain embodiments, the cancer, malignancy or dysproliferative change (such as a metaplasia or dysplasia), or hyperproliferative disorder, that induces anemia is of the ovary, breast, colon, lung, skin, pancreas, prostate, bladder, or uterus. In other specific embodiments, anemia induced by sarcoma, melanoma, or leukemia is treated or prevented. In certain other embodiments, the Triheterocyclic Compounds are used to treat or prevent anemia induced by chronic lymphocytic leukemia or myelodysplastic syndromes.

In certain embodiments, the subject in need of treatment or prevention of anemia has cancer, such as a cancer or neoplastic disease disclosed above. In other certain embodiments, the subject in need of treatment or prevention of anemia is also in need of treatment or prevention of cancer, such as described above.

In certain embodiments, the Triheterocyclic Compounds are used to treat or prevent anemia induced by one or more cancers including prostate (such as hormone-insensitive), neuroblastoma, lymphoma (such as follicular or Diffuse Large B-cell), breast (such as estrogen-receptor positive), colorectal, endometrial, ovarian, lymphoma (such as non-Hodgkin's), lung (such as small cell), or testicular (such as germ cell).

In certain embodiments, a Triheterocyclic Compound may be used where there is a possibility of anemia in an otherwise healthy individual such as an anticipated loss of blood during surgery. Accordingly, the invention encompasses methods for treating or preventing anemia that would result from blood loss due to surgery, comprising administering an effective amount of a Triheterocyclic Compound to a subject in need thereof.

In certain embodiments, treatment with a Triheterocyclic Compound may also be used for expediting hematopoietic recovery after acute blood loss.

### 5.11. Treatment or Prevention of Thrombocytopenia

In certain embodiments, the Triheterocyclic Compound can be used in the treatment or prevention of thrombocytopenia associated with, not limited to, a viral or bacterial infection; drug-induced thrombocytopenia (immune and non-immune); radiation-induced thrombocytopenia; aplastic anemia; cancer-induced thrombocytopenia, where the cancer is such as, but not limited to, breast, prostate, lung, skin, colorectal, cervical, pancreatic, ovarian, lymphoma, leukemia, liver, testicular, and brain cancer; idiopathic thrombocytopenic purpura; thrombotic thrombocytopenic purpura; hemolytic uremic syndrome; and hypersplenism.

In certain embodiments, the Triheterocyclic Compound can be used in the treatment or prevention of thrombocytopenia induced by one or more cancers or neoplastic disorders disclosed in §5.10, above.

In certain embodiments, the cancer, malignancy or dysproliferative change (such as a metaplasia or dysplasia), or hyperproliferative disorder that induces thrombocytopenia is of the ovary, breast, colon, lung, skin, pancreas, prostate, bladder, or uterus. In other specific embodiments, thrombocytopenia associated with sarcoma, melanoma, or leukemia is treated or prevented. In certain other embodiments, the Triheterocyclic Compounds are used to treat or prevent thrombocytopenia induced by chronic lymphocytic leukemia or myelodysplastic syndromes.

In certain embodiments, the Triheterocyclic Compounds are used to treat or prevent thrombocytopenia induced by one or more cancers including prostate (such as hormone-insensitive), neuroblastoma, lymphoma (such as follicular or Diffuse Large B-cell), breast (such as estrogen-receptor positive), colorectal, endometrial, ovarian, lymphoma (such as non-Hodgkin's), lung (such as small cell), or testicular (such as germ cell).

In certain embodiments, the subject in need of treatment or prevention of thrombocytopenia has cancer, such as a cancer or neoplastic disease disclosed in §5.10 above. In other certain embodiments, the subject in need or prevention of thrombocytopenia is also in need of treatment or prevention of cancer, such as described above.

In certain embodiments, a Triheterocyclic Compound may be used where there is a possibility of thrombocytopenia in an otherwise healthy individual such as an anticipated loss of blood during surgery. Accordingly, the invention encompasses methods for treating or preventing thrombocytopenia that would result from blood loss due to surgery, comprising administering an effective amount of a Triheterocyclic Compound to a subject in need thereof.

In certain embodiments, treatment with a Triheterocyclic Compound may also be used for expediting hematopoietic recovery after acute blood loss.

### 5.12. Other Therapeutic Agents

In certain embodiments, for the methods for treating or preventing anemia or thrombocytopenia can further comprise administering another therapeutic agent. With respect to anemia, such other therapeutic agents include, but are not limited to, antibodies (anti-thymocyte globulin (ATG), anti-lymphocyte globulin), immune-suppressing drugs (cyclosporine), cyclophosphamide, vincristine, corticosteroids, androgens, chemotherapy, synthetic erythropoietin, butyric acid, antifungal medication (clotrimazole), nitric oxide, pain relieving medication (ibuprofen) and supplemental oxygen.

Other therapeutic agents for thrombocytopenia include, but are not limited to, antibiotics, chemotherapy, intranasal desmopressin (ddAVP), radiation, immuno-suppressive drugs (for example, glucocorticoids, cytostatics, interferons, opioids, and antithymocyte globulin (ATG)).

In another embodiment, the present methods can further comprise performing a blood or platelet transfusion or a bone marrow transplant.

In certain embodiments, the present methods for treating or preventing anemia or thrombocytopenia further comprise administering another therapeutic agent to treat the underlying disease (for example, cancer, a viral infection, and lupus) of the anemia and/or thrombocytopenia.

In another embodiment, the other therapeutic agent can be another anti-cancer, chemotherapeutic agent including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. In another embodiment, the anti-cancer agent is one or more of those presented below in Table 2.

TABLE 2

| | |
|---|---|
| Radiation: | γ-radiation |
| Radiation Therapy enhancer: | Efaproxiral Sodium |
| | Motexafin Gadolinium |
| Alkylating agents | Mechlorethamine |
| | Melphalan |
| | Procarbazine |
| | Streptozocin |
| | Temozolomide |
| | Thiotepa |
| | Porfiromycin |
| | Altretamine |
| Nitrogen mustards: | cyclophosphamide |
| | Ifosfamide |
| | Trofosfamide |
| | Chlorambucil |
| | Bendamustine |
| Nitrosoureas: | carmustine (BCNU) |
| | Lomustine (CCNU) |
| | Estramustine |
| | Fotemustine |
| | Nimustine |
| | Ranimustine |
| Alkylsulphonates | Busulfan |
| | Treosulfan |
| Triazenes: | Dacarbazine |
| Platinum containing compounds: | Cisplatin |
| | Carboplatin |
| | Nedaplatin |
| | Oxaliplatin |
| Plant Alkaloids | Homoharringtonine |
| Vinca alkaloids: | Vincristine |
| | Vinblastine |
| | Vindesine |
| | Vinorelbine |
| | Vinoflunine |
| Taxoids: | Paclitaxel |
| | Docetaxol |
| DNA Topoisomerase Inhibitors | Amsacrine |
| | Dexrazoxane |
| Epipodophyllins: | Etoposide |
| | Teniposide |
| | Topotecan |
| | 9-aminocamptothecin |
| | irinotecan |
| | crisnatol |
| | Nitrocamptothecin |
| | Camptothecin |
| | CKD-602 |
| | Sobuzoxane |
| | Elinafide |
| Anti-metabolites | Thioguanine |
| | Cytarabine |
| | Tegafur |
| | Pentostatin |
| | Gemcitabine |
| | Capecitabine |
| Anti-folates: | Nolatrexed dihydrochloride |
| | Pemetrexed disodium |
| DHFR inhibitors: | Methotrexate |
| | Trimetrexate |
| IMP dehydrogenase Inhibitors: | mycophenolic acid |
| | Tiazofurin |
| | Ribavirin |
| | EICAR |
| Ribonuclotide reductase Inhibitors: | Hydroxyurea |
| | Deferoxamine |
| Pyrimidine analogs: | |
| Uracil analogs | 5-Fluorouracil |
| | Floxuridine |
| | Doxifluridine |
| | Ratitrexed |
| Cytosine analogs | cytarabine (ara C) |
| | Cytosine arabinoside |
| | Fludarabine |
| Nucleoside analogs | Troxacitabine |
| Purine analogs: | mercaptopurine |
| | Thioguanine |
| | Clofarabine |
| | Fludarabine phosphate |
| Hormonal therapies: | Estramustine |
| Receptor antagonists: | |
| Anti-estrogens | Tamoxifen |
| | Raloxifene |
| | Megestrol |
| Anti-androgens | Flutamide |
| | Bicalutamide |
| | Nilutamide |
| EGFR antagonist | Erlinotib |
| Estrogen receptor modifier: | Arzoxifene |
| Androgens | Fluoxymesterone |
| Progestational agent | Medroxyprogesterone Acetate |
| LHRH agonists: | Goserelin |
| | Leuprolide acetate |
| | Triptorelin pamoate |
| Retinoids/Deltoids | |
| Vitamin D3 analogs | EB 1089 |
| | CB 1093 |
| | KH 1060 |
| Vitamin A derivative | Isotretinoin |
| | Tretinoin |
| Retinoid | Bexarotene |
| Photodyamic therapies: | Vertoporfin (BPD-MA) |
| | Phthalocyanine |
| | photosensitizer Pc4 |
| | Demethoxy-hypocrellin A |
| | (2BA-2-DMHA) |
| Cytokines: | Interferon-α |
| | Interferon-γ |
| | Interferon-β |
| | Tumor necrosis factor |
| Others: | Cladribine |
| | Exisulind |
| | Fenretimide |
| | Irofulven |
| | Leucovorin calcium |
| | Mitotane |
| | ONYX-015 |
| | Prednisone |
| | Raltitrexed |
| | Suramin |
| | Thalidomide |
| | Tipifarnib |
| | Tirapazamide |
| | Toremifene |
| Enzyme | Asparaginase |
| Isoprenylation inhibitors: | Lovastatin |
| Dopaminergic neurotoxins: | 1-methyl-4-phenylpyridinium ion |
| Kinase inhibitors: | Staurosporine |
| | Imatinib mesylate |
| | Gefitinib |
| | Bryostatin-1 |
| | Flavopridol |
| | Erlotinib |
| | Isis 3521 |
| Proteosome inhibitors: | Bortezomib |
| | PS-341 |
| Aromatase inhibitors: | Aminoglutethemine |
| | Anastrozole |
| | Exemestane |
| | Letrozole |
| Antibiotics: | Mitoxantrone |
| | Plicamycin |
| Actinomycins | Actinomycin D |
| | Dactinomycin |
| Mytomycins | Mytomycin C |
| Bleomycins: | Bleomycin A2 |
| | Bleomycin B2 |
| | Peplomycin |
| Anthracyclines: | Daunorubicin |
| | Doxorubicin (adriamycin) |
| | Idarubicin |
| | Epirubicin |
| | Pirarubicin |
| | Zorubicin |
| | Mitoxantrone |
| | Valrubicin |
| | Amrubicin |
| Antibodies: | Trastuzumab |
| | Bevacizumab |
| | Alemtuzumab |
| | Gemtuzumab ozogamicin |
| | Daclizumab |
| | Edrecolomab |
| | Tositumomab, iodine I131 |
| | Muromonab-CD3 |
| | Ibritumomab tiuxetan |
| | Rituximab |
| | Cetuximab |
| Vaccine: | CEA vaccine |
| | HSPPC-96 |
| | Melanoma theraccine |
| MDR inhibitors | Verapamil |
| Antiangiogenic agents: | AE-941 |
| | Arsenic trioxide |
| Ca²⁺ ATPase inhibitors: | Thapsigargin |

In another embodiment, an effective amount of a Triheterocyclic Compound is administered concurrently with another therapeutic agent, chemotherapy or radiation therapy. In another specific embodiment, the therapeutic agent, chemotherapy or radiation therapy is administered immediately prior or immediately subsequent to administration of a Triheterocyclic Compound.

If the Triheterocyclic Compound is administered prior to administering another therapeutic agent, chemotherapy or radiation therapy, the other therapeutic agent, chemotherapy or radiation therapy is administered while the Triheterocyclic Compound is exerting its therapeutic or prophylactic effect. If the other therapeutic agent, chemotherapy or radiation therapy is administered prior to administering a Triheterocyclic Compound, the Triheterocyclic Compound is administered while the other therapeutic agent, chemotherapy or radiation therapy is exerting its therapeutic effect.

The other therapeutic agent or chemotherapeutic agents can be administered in a series of sessions, any one or a combination of the other therapeutic agents or chemotherapeutic agents listed above can be administered. With respect to radiation therapy, any radiation therapy protocol can be used depending upon the type of cancer to be treated. For example, but not by way of limitation, x-ray radiation can be administered; in particular, high-energy megavoltage (radiation of greater that 1 MeV energy) can be used for deep tumors, and electron beam and orthovoltage x-ray radiation can be used for skin cancers. Gamma-ray emitting radioisotopes, such as radioactive isotopes of radium, cobalt and other elements, may also be administered to expose tissues to radiation.

### 6. EXAMPLES

### Example 1

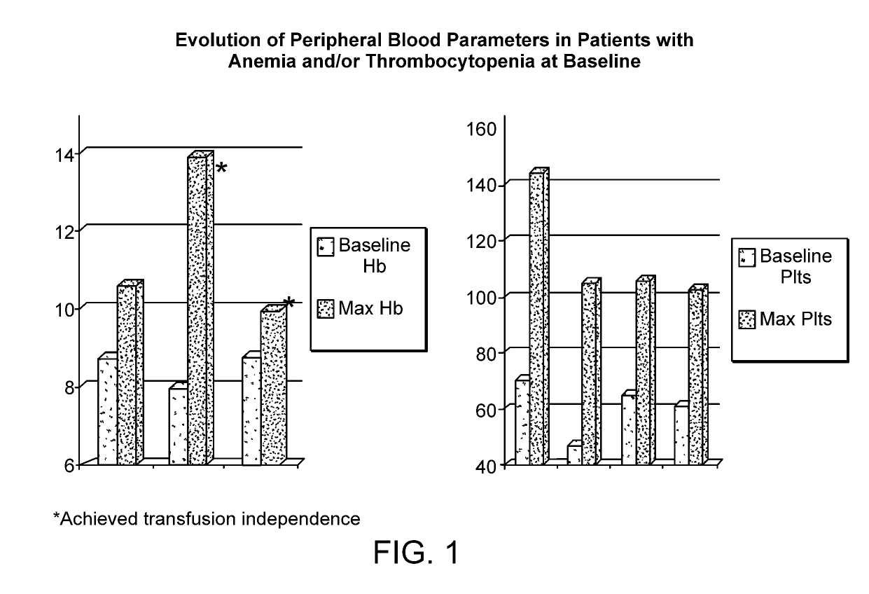

In a multi-center, double-blind, dose-ranging study, two groups of patients having chronic lymphocytic leukemia (CLL) received intravenous doses of either 3.5-14 mg/m² of the mesylate salt of Compound 1 administered as a 1-hour infusion, or a 20-40 mg/m2 dose via a 3-hour infusion. These infusions were administered over a period of several weeks to several months. Four patients out of fourteen patients having thrombocytopenia (with initial platelet counts less than 150×10⁹/L) showed significant increases of over 50% in platelet count after administration of the mesylate salt of Compound 1. In addition, three out of eleven anemic patients (with initial hemoglobin counts of less than 11 g/dL) showed significant hemoglobin elevation after administration of the mesylate salt of Compound 1, with two of the patients becoming transfusion independent (see FIG. 1 for results).

These data demonstrate that Compound 1, an illustrative Triheterocyclic Compound, is useful for treating anemia and/or thrombocytopenia.

### Example 2

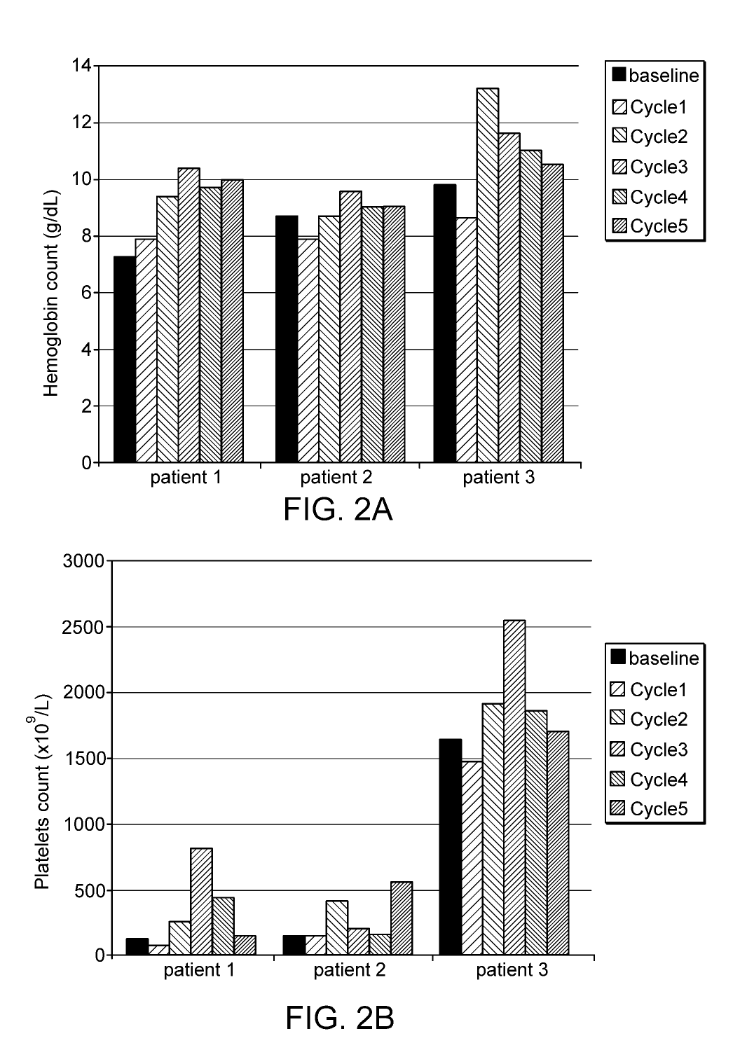

In a multi-center, open label, dose escalation study, a group of eight patients having myelodysplastic syndromes (MDS) received intravenous doses of 7-40 mg/m² of the mesylate salt of Compound 1 administered as a 24-hour infusion every two weeks. These infusions were administered over a period of several weeks to several months, the first cycle of infusion was administered in a clinical unit but the subsequent four cycles were administered in the ambulatory setting using portable infusion pumps. Three out of eight patients having MDS showed hematological improvement in hemoglobin levels after administration of the mesylate salt of Compound 1, with two of the patients becoming transfusion independent (see FIG. 2A for results). In addition, these three patients showed platelet count elevation after administration of the mesylate salt of Compound 1, with one of the patients becoming transfusion independent (see FIG. 2B for results). The platelet count at the start of the study for patient 3 in FIG. 2B indicates that this patient was not thrombocytopenic.

These data demonstrate that Compound 1, an illustrative Triheterocyclic Compound, is useful for treating anemia and/or thrombocytopenia.

The present invention is not to be limited in scope by the specific embodiments disclosed in the examples, which are intended as illustrations of a few aspects of the invention, and any embodiments that are functionally equivalent are within the scope of this invention. Indeed, various modifications of the invention in addition to those shown and described herein will become apparent to those skilled in the art and are intended to fall within the scope of the appended claims.

What is claimed is:

1. A method for treating or preventing anemia or thrombocytopenia, comprising administering to a subject in need thereof an effective amount of a compound of Formula (Ia) 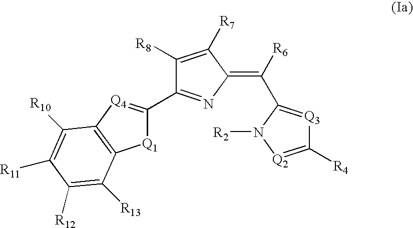
or a pharmaceutically acceptable salt thereof; wherein
Q₁ is —O—, —S— or —N(R₁)—;
Q₂ is —C(R₃)— or —N—;
Q₃ is —C(R₅)— or —N—;
Q₄ is —C(R₉)— or —N—;
R₁ is —Yₘ(Rₐ), wherein —Rₐ is —H, —OH, —C₁-C₈ alkyl, —C₂-C₈ alkenyl, —C₂-C₈ alkynyl, —C₃-C₁₂ cycloalkyl, -phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —OS(O)₂OH, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, or —NR₁₄C(S)N(R₁₄)₂;
R₂ is —H, —C₁-C₈ alkyl or —OH;
R₃, R₄, and R₅ are independently —Yₘ(R_{b}), wherein R_{b} is —H, halogen, —NH₂, —CN, —NO₂, —SH, —N₃, —C₁-C₈ alkyl, —O—(C₁-C₈ alkyl), —C₂-C₈ alkenyl, —C₂-C₈ alkynyl, —C₃-C₁₂ cycloalkyl, -phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙR₁₄, —O(CH₂)ₙ—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSOR₁₄, —NHS(O)₂R₁₄, O—C(S)R₁₄, O—C(S)OR₁₄, O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R,₄, —NHC(S)NHR₁₄, —NHC(S)N(R₁₄)₂, —NR₁₄C(S)NHR₁₄, —NR₁₄C(S)N(R₁₄)₂ or R₃ and R₄, or R₄ and R₅, together with the carbon atom to which each is attached, join to form a 5- to 9-membered ring, with the proviso that if Q₃ is —C(R₅)— and m=0, then R₅ is not H;
R₆ is —H;
R₇ is —Yₘ—(R_{c}), wherein —R_{c} is —C₁-C₈ alkyl, —O—(C₁-C₈ alkyl), —O-benzyl, —OH, —NH₂, —NH(C₁-C₅ alkyl), —N(C₁-C₅ alkyl)₂, —NH(phenyl), —N(phenyl)₂, —NH(naphthyl), —N(naphthyl)₂, —CN, —NO₂, —N₃, —C₂-C₈ alkynyl, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O(CH₂)ₙC(O)O(CH₂)ₙCH₃, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂;
R₈ is —Yₘ(R_{d}), wherein —R_{d} is —H, —OH, halogen, —NH₂, —NH(C₁-C₅ alkyl), —N(C₁-C₅ alkyl)₂, —NH(phenyl), —N(phenyl)₂, —NH(naphthyl), —N(naphthyl)₂, —CN, —NO₂, —N₃, —C₁-C₈ alkyl, —O—(C₁-C₈ alkyl), —(C₁-C₈ alkylene)-OH, —C₂-C₈ alkenyl, —C₂-C₈ alkynyl, —C₃-C₁₂ cycloalkyl, -phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂;
R₉, R₁₀, R₁₁, R₁₂, and R₁₃ are independently —Yₘ(Rₑ), wherein —Rₑ is —H, halogen, —NH₂, C₁-C₈ alkyl, —NH(C₁-C₅ alkyl), —N(C₁-C₅ alkyl)₂, —NH(C₂-C₅ alkenyl), —N(C₂-C₅ alkenyl)₂, —NH(phenyl), —N(phenyl)₂, —NH(naphthyl), —N(naphthyl)₂, —C(O)NH(C₁-C₅ alkyl), —C(O)N(C₁-C₅ alkyl)₂, —NHC(O)(C₁-C₅ alkyl), —NHC(═NH₂⁺)NH₂, —CN, —NO₂, N₃, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, C(O)C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂ or R₁₁ and R₁₂, together with the carbon atom to which each is attached, join to form a 5- to 9-membered ring;
each R₁₄ is independently —H, —C₁-C₈ alkyl, —C₃-C₁₂ cycloalkyl, -phenyl, -naphthyl, -3- to 9-membered heterocycle, —C₂-C₈ alkenyl, or —C₂-C₈ alkynyl; each Y is independently —C₁-C₈ alkylene-, —C₂-C₈ alkenylene- or —C₂-C₈ alkynylene-;
each m is independently 0 or 1;
each n is independently an integer ranging from 0 to 6; and
each p is independently an integer ranging from 1 to 6.

2. The method of claim 1, further comprising administering another therapeutic agent.

3. The method of claim 1, wherein the pharmaceutically acceptable salt is a tartrate salt or a mesylate salt.

4. A method for treating or preventing anemia or thrombocytopenia, comprising administering to a subject in need thereof an effective amount of a compound of Formula (Ib) 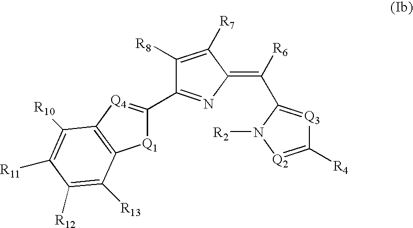
or a pharmaceutically acceptable salt thereof;
wherein
Q₁ is —O—, —S—or —N(R₁)—;
Q₂ is —C(R₃)— or —N—;
Q₃ is —C(R₅)— or —N—;
Q₄ is —C(R₉)— or —N—;
R₁ is —Yₘ(Rₐ), where Rₐ is selected from —H, —OH, —C₁-C₈ alkyl, —C₂-C₈ alkenyl, —C₂-C8 alkynyl, —C₃-C₁₂ cycloalkyl, -phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —OS(O)₂OH, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, or —NR₁₄C(S)N(R₁₄)₂;
R₂ is —H, —C₃-C₈ alkyl or —OH;
R₃, R₄, and R₅ are independently —Yₘ—(R_{b}), wherein R_{b} is —H, halogen, —NH₂, —CN, —NO₂, —SH, —N₃, —C₁-C₈ alkyl, —O—(C₁-C₈ alkyl), —C₂-C₈ alkenyl, —C₂-C₈ alkynyl, —C₃-C₁₂ cycloalkyl, -phenyl, -naphthyl, -3- to 9-membered heterocycle, —OR₁₄, —O(CH₂)ₚOR₁₄, —O—C(O)R₁₄, —C(O)(CH₂)ₙ—R₁₄, —C(O)R₁₄, —O(CH₂)ₙ—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂ or R₃ and R₄, or R₄ and R₅ together with the carbon atom to which each is attached, join to form a 5- to 9-membered ring, with the proviso that if Q₃ is —C(R₅)— and m=0, then R₅ is not H;
R₆ is —H, halogen, —OH, —NH₂, —C₁-C₈ alkyl, or —O—(C₁-C₈ alkyl);
R₇ and R₈ are independently —Yₘ(R_{d}) wherein R_{d} is —H, —OH, halogen, —NH₂, —NH(C₁-C₅ alkyl), —N(C₁-C₅ alkyl)₂, —NH(phenyl), —N(phenyl)₂, —NH(naphthyl), —N(naphthyl)₂, —CN, —NO₂, —N₃, —C₁-C₈ alkyl, —O—(C₁-C₈ alkyl), —(C₁-C₈ alkylene)-OH, —O-benzyl, —C₂-C₈ alkenyl, —C₂-C₈ alkynyl, —C₃-CI₂ cycloalkyl, -phenyl, -naphthyl, -3- to 9-membered heterocycle, —OR₁₄, —CH₂O(CH₂)ₚOR₁₄, —O—C(O)R₁₄, —C(O)(CH₂)ₙ—R₁₄, —C(O)R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂;
R₉, R₁₀, R₁₁, R₁₂, and R₁₃ are independently —Yₘ(Rₑ) wherein Rₑ is —H, halogen, —NH₂, C₁-C₈ alkyl, —NH(C₁-C₅ alkyl), —N(C₁-C₅ alkyl)₂, —NH(C₂-C₅ alkenyl), —N(C₂-C₅ alkenyl)₂, —NH(phenyl), —N(phenyl)₂, —NH(naphthyl), —N(naphthyl)₂, —C(O)NH(C₁-C₅ alkyl), —C(O)N(C₁-C₅ alkyl)₂, —NHC(O)(C₁-C₅ alkyl), —NHC(═NH₂⁺)NH₂, —CN, —NO₂, N₃, -3- to 9-membered heterocycle, —OR₁₄, —CH₂O(CH₂)ₚOR₁₄, —O—C(O)R₁₄, —C(O)(CH₂)ₙ—R₁₄, —C(O)R₁₄, —O—C(O)OR₁₄, —C(O)C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂ or R₁₁ and R₁₂, together with the carbon atom to which each is attached, join to form a 5- to 9-membered ring; and
each R₁₄ is independently —H, —C₁-C₈ alkyl, —C₃-C₁₂ cycloalkyl, -phenyl, -naphthyl, -3- to 9-membered heterocycle, —C₂-C₉ alkenyl, or —C₂-C₈ alkynyl;
each Y is independently —C₁-C₈ alkylene-, —C₂-C₈ alkenylene- or —C₂-C₈ alkynylene-;
each m is independently 0 or 1;
each n is independently an integer ranging from 0 to 6; and
each p is independently an integer ranging from 1 to 6.

5. The method of claim 4, further comprising administering another therapeutic agent.

6. The method of claim 4, wherein the pharmaceutically acceptable salt is a tartrate salt or a mesylate salt.

7. A method for treating or preventing anemia or thrombocytopenia, comprising administering to a subject in need thereof an effective amount of the compound or a pharmaceutically acceptable salt of the compound having the formula: 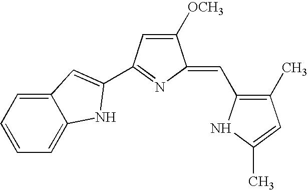
or a pharmaceutically acceptable salt thereof.

8. The method of claim 7, further comprising administering another therapeutic agent.

9. The method of claim 7, wherein the pharmaceutically acceptable salt is a tartrate salt or a mesylate salt.

10. A method for treating or preventing anemia or thrombocytopenia, comprising administering to a subject in need thereof an effective amount of a compound of Formula (Ic) 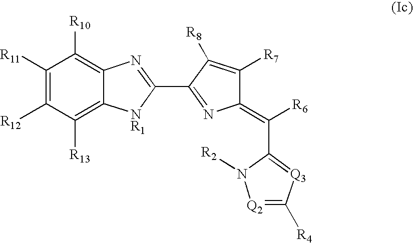
or a pharmaceutically acceptable salt thereof, wherein:
Q₂ is —C(R₃)— or —N—;
Q₃ is —C(R₅)— or —N—;
R₁ is —Yₘ(Rₐ), wherein —Rₐ is —H, —OH, —C₁-C₈ alkyl, —C₂-C₈ alkenyl, —C₂-C₈ alkynyl, —C₃-C₁₂ cycloalkyl, -phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —OS(O)₂OH, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, or —NR₁₄C(S)N(R₁₄)₂;
R₂ is —H, —C₁-C₈ alkyl or —OH;
R₃, R₄, and R₅ are independently —Yₘ(R_{b}), wherein R_{b} is —H, halogen, —NH₂, —CN, —NO₂, —SH, —N₃, —C₁-C₈ alkyl, —O—(C₁-C₈ alkyl), —C₂-C₈ alkenyl, —C₂-C₈ alkynyl, —C₃-C₁₂ cycloalkyl, -phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O(CH₂)ₙ—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂ or R₃ and R₄, or R₄ and R₅ together with the carbon atom to which each is attached, join to form a 5- to 9-meinbered ring;
R₆ is —H, halogen, —OH, —NH₂, —C₁-C₈ alkyl, or —O—(C₁-C₈ alkyl);
R₇ is —Yₘ—(R_{c}), wherein —R_{c} is —C₁-C₈ alkyl, —O—(C₁-C₈ alkyl), —O-benzyl, —OH, —NH₂, —NH(C₁-C₅ alkyl), —N(C₁-C₅ alkyl)₂, —NH(phenyl), —N(phenyl)₂, —NH(naphthyl), —N(naphthyl)₂, —CN, —NO₂, —N₃, —C₂-C₈ alkynyl, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O(CH₂)ₙC(O)O(CH₂)ₙCH₃, —O—C(S)R₁₄, —O—C(S)OR₁₄, —O—C(S)NHR₁₄, O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂;
R₈ is —Yₘ(R_{d}), wherein —R_{d} is —H, —OH, halogen, —NH₂, —NH(C₁-C₅ alkyl), —N(C₁-C₅ alkyl)₂, —NH(phenyl), —N(phenyl)₂, —NH(naphthyl), —N(naphthyl)₂, —CN, —NO₂, —N₃, —C₁-C₈ alkyl, —O—(C₁-C₈ alkyl), —(C₁-C₈ alkylene)-OH, —C₂-C₈ alkenyl, —C₂-C₈ alkynyl, —C₃-C₁₂ cycloalkyl, -phenyl, -naphthyl, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂;
R₁₀, R₁₁, R₁₂, and R₁₃ are independently —Yₘ(Rₑ), wherein —Rₑ is —H, halogen, —NH₂, C₁-C₈ alkyl, —NH(C₁-C₅ alkyl), —N(C₁-C₅ alkyl)₂, —NH(phenyl), —N(phenyl)₂, —NH(naphthyl), —N(naphthyl)₂, —C(O)NH(C₁-C₅ alkyl), —C(O)N(C₁₋₅-C₅ alkyl)₂, —NHC(O)(C₁-C₅ alkyl), —NHC(═NH₂⁺)NH₂, —CN, —NO₂, N₃, -3- to 9-membered heterocycle, —OR₁₄, —O(CH₂)ₚOR₁₄, —C(O)R₁₄, —O—C(O)R₁₄, —C(O)(CH₂)ₙ—R₁₄, —O—C(O)OR₁₄, —O—C(O)NHR₁₄, —O—C(O)N(R₁₄)₂, —C(O)N(R₁₄)₂, —C(O)OR₁₄, —C(O)NHR₁₄, —S—R₁₄, —SOR₁₄, —S(O)₂R₁₄, —NHC(O)R₁₄, —NHSR₁₄, —NHSOR₁₄, —NHS(O)₂R₁₄, —O—C(S)R₁₄, —O—C(S)OR₁₄, —O—C(S)NHR₁₄, —O—C(S)N(R₁₄)₂, —C(S)OR₁₄, —C(S)NHR₁₄, —C(S)N(R₁₄)₂, —NHC(S)R₁₄, —NR₁₄C(S)R₁₄, —NHC(S)NHR₁₄, —NHC(S)N(R₁₄)₂, —NR₁₄C(S)NHR₁₄, —NR₁₄C(S)N(R₁₄)₂; or R₁₁ and R₁₂ together with the carbon atom to which each is attached, join to form a 5- to 9-membered ring;
each R₁₄ is independently —H, —C₁-C₈ alkyl, —C₃-C₁₂ cycloalkyl, -phenyl, -naphthyl, -3- to 9-membered heterocycle, —C₂-C₈ alkenyl, or —C₂-C₈ alkynyl;
each Y is independently —C₁-C₈ alkylene-, —C₂-C₈ alkenylene- or —C₂-C₈ alkynylene-;
each m is independently 0 or 1;
each n is independently an integer ranging from 0 to 6; and
each p is independently an integer ranging from 1 to 6.

11. The method of claim 10, further comprising administering another therapeutic agent.

12. The method of claim 10, wherein the pharmaceutically acceptable salt is a tartrate salt or a mesylate salt.

13. The method of claim 1, wherein the preventing is in a subject having a heightened risk of developing anemia or thrombocytopenia.

14. The method of claim 13, further comprising administering another therapeutic agent.

15. The method of claim 13, wherein the pharmaceutically acceptable salt is a tartrate salt or a mesylate salt.

16. The method of claim 4, wherein the preventing is in a subject having a heightened risk of developing anemia or thrombocytopenia.

17. The method of claim 16, further comprising administering another therapeutic agent.

18. The method of claim 16, wherein the pharmaceutically acceptable salt is a tartrate salt or a mesylate salt.

19. The method of claim 7, wherein the preventing is in a subject having a heightened risk of developing anemia or thrombocytopenia.

20. The method of claim 19, further comprising administering another therapeutic agent.

21. The method of claim 19, wherein the pharmaceutically acceptable salt is a tartrate salt or a mesylate salt.

22. The method of claim 10, wherein the preventing is in a subject having a heightened risk of developing anemia or thrombocytopenia.

23. The method of claim 22, further comprising administering another therapeutic agent.

24. The method of claim 22, wherein the pharmaceutically acceptable salt is a tartrate salt or a mesylate salt.
